(19) United States
(12) Patent Application Publication
(10) Pub. No.: US 2013/0115133 A1
(43) Pub. Date: May 9, 2013

(54) INACTIVATION OF MICROORGANISMS WITH MULTIDRUG RESISTANCE INHIBITORS AND PHENOTHIAZINIUMS

(71) Applicants: Michael R. Hamblin, Revere, MA (US); George P. Tegos, Boston, MA (US)

(72) Inventors: Michael R. Hamblin, Revere, MA (US); George P. Tegos, Boston, MA (US)

(73) Assignee: THE GENERAL HOSPITAL CORPORATION, Boston, MA (US)

(21) Appl. No.: 13/527,412

(22) Filed: Jun. 19, 2012

Related U.S. Application Data
(63) Continuation of application No. 11/662,977, filed on Sep. 26, 2007, now abandoned.
Application No. 11/662,977 is a continuation of application No. PCT/US2005/033523, filed on Sep. 19, 2005.
(60) Provisional application No. 60/610,708, filed on Sep. 17, 2004.

(51) Int. Cl. A61K 41/00; A61N 5/06; A01N 43/84; A61K 31/7028; A61K 38/05

(52) U.S. Cl. CPC A61K 41/0057; A61K 31/7028; A61K 38/05; A01N 43/84; A61N 5/062
USPC 422/29; 514/224.8; 514/25; 514/2.3; 604/20

(57) ABSTRACT

The present invention relates the use of phenothiaziniums and microbial MDR inhibitors to inactivate microorganisms. Methods of the present invention are useful in the treatment of living subjects and in the decontamination of inanimate objects and substances.

### RELATED APPLICATIONS/PATENTS & INCORPORATION BY REFERENCE

This application is a continuation of U.S. Utility application Ser. No. 11/662,977, filed on Sep. 26, 2007 which is the U.S. national phase, pursuant to 35 U.S.C. §371, of International Patent Application No. PCT/US2005/033523, filed on Sep. 19, 2005, designating the U.S. and published in English on Mar. 30, 2006 as International Publication No. WO 2006/034219 A2 which claims priority to U.S. Provisional Application Serial No. 60/610,708, filed on Sep. 17, 2004, the entire disclosures of all of which are incorporated herein by reference.

Each of the applications and patents cited in this text, as well as each document or reference cited in each of the applications and patents (including during the prosecution of each issued patent; “application cited documents”), and each of the PCT and foreign applications or patents corresponding to and/or claiming priority from any of these applications and patents, and each of the documents cited or referenced in each of the application cited documents, are hereby expressly incorporated herein by reference, and may be employed in the practice of the invention. More generally, documents or references are cited in this text, either in a Reference List before the claims, or in the text itself; and, each of these documents or references (“herein cited references”), as well as each document or reference cited in each of the herein cited references (including any manufacturer's specifications, instructions, etc.), is hereby expressly incorporated herein by reference.

### STATEMENT OF GOVERNMENT SUPPORT

This invention was made with Government Support under Grant No. AI050875 awarded by the National Institutes of Health. The Government has certain rights in this invention

### BACKGROUND OF THE INVENTION

Efflux mechanisms have become broadly recognized as major components of resistance to many classes of antibiotics. Some efflux pumps selectively extrude specific antibiotics while others, referred to as Multidrug Resistance Pumps (MDRs) expel a variety of substrates, including structurally diverse compounds with differing modes of action. Based on sequence similarity, multidrug transporter systems are classified into six super-families i) ATP Binding Cassette Transporters (ABC), ii) Major Facilitators (MF), iii) Resistance Nodulation Division (RND), iv) Small Multi-drug Resistance (SMR) v) Multi-drug And Toxic Compound Extrusion (MATE) and vi) Multi-drug Endosomal Transporter (MET) family (Paulsen, 2002). Multi-drug resistant human pathogenic microorganisms are directly associated with serious recalcitrant infections such as cystic fibrosis, nosocomial infections and infections in immunocompromised patients undergoing anticancer chemotherapy or infected with HIV.

Photodynamic therapy, which involves the use of photoactivatable compounds to produce toxic effects in cells, has been used to target and destroy microorganisms. Phenothiaziniums represent one such class of photoactivatable compounds. Phenothiaziniums were not known or suspected to be substrates for MDRs.

### SUMMARY OF THE INVENTION

It has now been shown that phenothiaziniums are substrates for microbial Multidrug Resistance Pumps (“MDR Pumps”). Inactivation of the microbial MDR Pumps by inhibitors (“MDR inhibitors”) increases the amount of phenothiazinium that can be retained by the microorganism, thereby increasing efficacy upon photoactivation of the phenothiazinium by irradiation.

Accordingly, in one aspect, the present invention provides a method of inactivating microorganisms comprising contacting the microorganism with a phenothiazinium and a microbial MDR inhibitor and irradiating the phenothiazinium such that a phototoxic species is produced that inactivates the microorganism.

The microorganism can be contacted with the phenothiazinium and the microbial MDR inhibitor sequentially (in either order) or at the same time. In one embodiment, the phenothiazinium and the microbial MDR inhibitor can be formulated in the same pharmaceutical composition.

Phenothiaziniums include but are not limited to toluidine blue derviatives, toluidine blue O (TBO), methylene blue (MB), new methylene blue N (NMMB), new methylene blue BB, new methylene blue FR, 1,9-dimethylmethylene blue chloride (DMMB), methylene blue derivatives, methylene green, methylene violet Bernthsen, methylene violet 3RAX, Nile blue, Nile blue derivatives, malachite green, Azure blue A, Azure blue B, Azure blue C, safranin O, neutral red, 5-ethylamino-9-diethylaminobenzo[a]phenothiazinium chloride, 5-ethylamino-9-diethylaminobenzo[a]phenoselenazinium chloride, thiopyronine, and thionine.

Microbial MDR inhibitors include but are not limited to INF271, MC₂₀₇₁₁₀, 5′ Methoxyhydnocarpin (“5-MHC”), Pheophorbide a, Chrysoplenol D, Chrysoplenetin, Genistein, Biochanin, Polyacylated Neohesperidosides, Polyacylated Neohesperidosides, 4′,6′-Dihydroxy-3′,5′ dimethyl-2′-methoxychalcone, 3,5-Dimethoxy-4′-hydroxy-trans-stilbene, 3,5,4′-Trimethoxy-trans-stilbene, Difluorocyclopropyl quinoline (“Zosuquidar 3HCL” or “LY335979”), Dihydropyrroloquinolines, GG918, Verpamil Pgp, Cyclosporins Pgp, Reserpine Pgp, Propafenone Pgp, Pyridazino[4,3-b]indoles Pgp, Hypericin, Cyclooxygenase-2 (“Cox-2”), 3-Oxopiperazinium and Perhydro-3-oxo-1,4-diazepinium derivatives, Tetrandrine, and Phenothiazines.

In one aspect, the present invention provides a method of treating a subject infected with a microorganism, said method comprising the steps of administering a phenothiazinium and a microbial MDR inhibitor to the subject, irradiating the phenothiazinium such that a phototoxic species is produced that inactivates the microorganism, thereby treating the subject.

In another aspect, the present invention provides methods for the inactivation of microorganisms found in inanimate substances and objects, such as animal-derived products, biological fluids, food, water, air, hard-surfaces, equipment, and machinery and clothing.

In a specific embodiment, the microorganism inhabits a biofilm.

In certain embodiments the phenothiaziniums and/or microbial MDR inhibitors of the present invention are formulated in compositions that also contain one or more additional agents such as pharmaceutically acceptable carriers, excipients, antibiotics, antimicrobial agents (e.g., bactericidal, antiviral or antifungal agents), disinfectants, or detergents. In other embodiments, phenothiaziniums and/or microbial MDR inhibitors of the present invention are co-administered with one or more additional agents such as antibiotics, antimicrobial agents (e.g., bactericidal, antiviral or antifungal agents), disinfectants, or detergents.

In specific embodiments, irradiation is provided by a light source that emits light having a wavelength in the range of about 450 to about 750 nm and/or with a fluence in the range of about 10 to about 1000 J/cm². Such a light source can be, for example, natural sunlight, a lamp, a laser or a fiber optic device.

Other objects and advantages of the present invention will be apparent from the detailed description that follows.

### BRIEF DESCRIPTION OF THE FIGURES

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings, in which:

FIG. 1 depicts multidrug resistance efflux pumps.

FIG. 2 depicts some of the microbial MDR inhibitors known in the art.

FIG. 3 depicts the chemical structures of some of the photosensitizers known in the art.

FIG. 4 depicts the phototoxicity of Methylene Blue (MB) after incubation at concentration of 10 μM by S. aureus NorA− wild type, and NorA+. Values are means of three separate experiments and bars are SEM. * P<0.05; ** P<0.01 *** P<0.001 compared to wild type.

FIG. 5 depicts the phototoxicity of MB after incubation at concentration of 50 μM by E. coli wild type and TolC−. Conditions were as described for FIG. 4.

FIG. 6 depicts the phototoxicity of MB after incubation at concentration of 300 μM by P. aeruginosa MexAB−, wild type and MexAB+. Conditions were as described for FIG. 4.

FIG. 7(a) depicts the phototoxicity of Toluidine Blue O (TBO) and FIG. 7(b) of DMMB after incubation at a concentration of 10 μM by S. aureus NorA−, wild type, and NorA+.

FIG. 8(a) depicts the phototoxicity of Rose Bengal (RB) at 10 μM and FIG. 8(b) of pL-ce6 at 1 μM both with a wash with S. aureus NorA−, wild type, and NorA+, followed by illumination with 100 mWcm⁻² 540-nm light for RB and 660-nm light for pL-ce6.

FIG. 9(a) depicts, in bar-graph form, the uptake of photosensitizer in terms of molecules per cell by S. aureus NorA−, wild type, and NorA+. Values are means of three separate determinations and bars are SEM. * P<0.05; ** P<0.01 compared to wild type. FIG. 9(b) depicts, in bar-graph form, the uptake of MB and TBO in terms of molecules per cell by E. coli TolC−, and wild type, and P. aeruginosa MexAB−wild type and MexAB+. Values are means of three separate determinations and bars are SEM. * P<0.05; ** P<0.01 *** P<0.001 compared to wild type.

FIG. 10 depicts the phototoxicity of MB after incubation at a concentration of 10 μM with or without 10 μg/ml neohesperidoside derivative (ADH7) by (a) S. aureus wild type, and (b) S. aureus NorA+.

FIG. 11 depicts the phototoxicity of TBO after incubation at a concentration of 250 μM by P. aeruginosa PAO1 with or without 10 μg/ml of the synthetic MDR inhibitor MC207110. Values are means of three separate experiments and bars are SEM. * P<0.05; ** P<0.01 *** P<0.001 compared to wild type

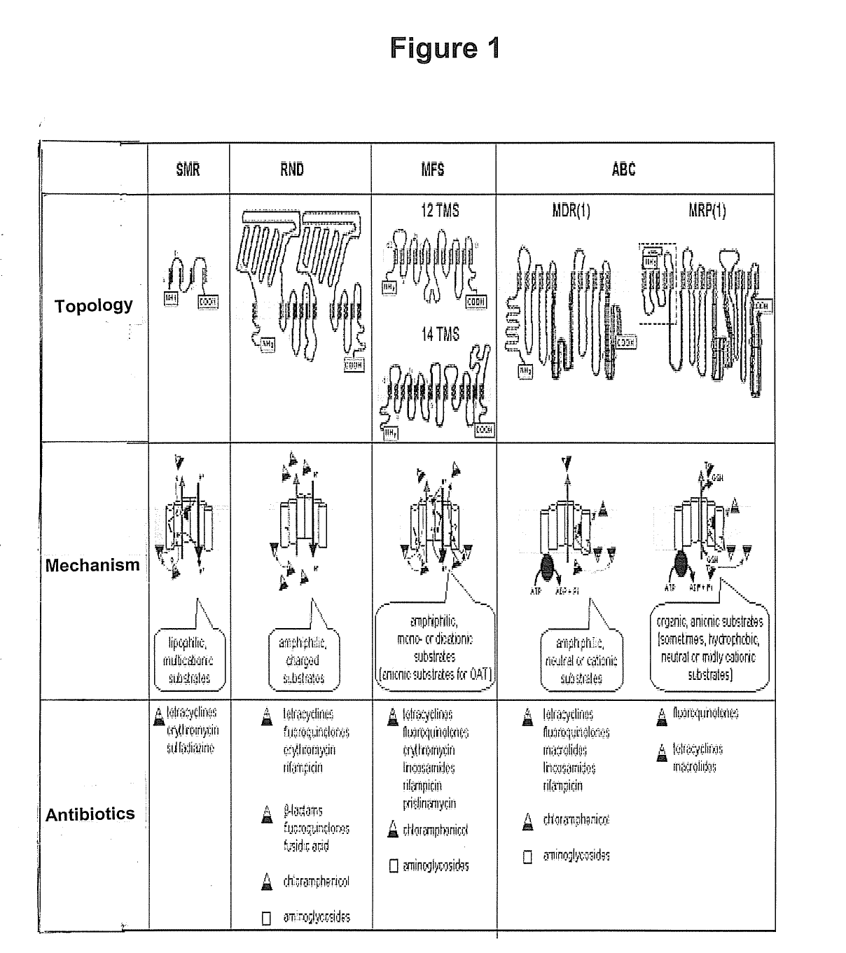

### DETAILED DESCRIPTION OF THE INVENTION

### I. Definitions

The term “microorganism” as used herein has its normal meaning which is well known and understood by those of skill in the art to refer to any microscopic organism. A microorganism can be, for example, a bacterium, fungus, protozoa, virus, parasite, yeast or an arthropod.

A “biofilm” refers to a colony of microorganisms which inhabit a common area and share biological resources (Stoodley, 2004).

“Inactivation” or “inactivating” as used herein refers to any method of killing, destroying, or otherwise functionally incapacitating a microorganism.

The term “decontaminate” as used herein refers to the process of inactivating microorganisms, and can be used interchangeably with the terms “disinfect” and “sterilize.” The terms “inanimate substance” and “inanimate object,” as used herein mean any material thing that is not a whole living animal, and includes materials comprising or consisting of solids, liquids and gases. “Substances” and “objects” can consist of or comprise living material such as plants and parts of animals such as isolated animal tissues or cells.

A “subject” is a vertebrate, preferably a mammal, more preferably a human. Mammals include, but are not limited to, humans, animals (farm animals, sport animals, and pets).

As used herein, the following terms have the meanings ascribed to them unless specified otherwise. As used herein, the terms “comprises”, “comprising”, and the like can have the meaning ascribed to them in U.S. Patent Law and can mean “includes”, “including” and the like.

Further definitions may appear in context throughout the disclosure provided herein.

### II. Methods of the Invention

Methods of the present invention provide a means for treating a subject that harbors, or is infected with a microorganism. Methods of the invention can be performed by contacting the subject with a phenothiazinium and a microbial MDR inhibitor and irradiating the phenothiazinium with a light source that emits light at an effective wavelength and fluence rate (i.e., an “effective light source”). In so doing, microorganism will be inactivated.

As used herein “treatment” refers to the application or administration of the phenothiazinium and the microbial MDR inhibitor followed by irradiation of the phenothiazinium with an effective light source. Treatment may be performed only once, or may be repeated as desired until the microorganism is inactivated. For example, successive treatments at hourly intervals may be used. Alternatively, treatments may be performed twice daily, or as directed by a physician.

If the microorganism, or infection produced by the microorganism, is suspected of being located at a particular location in or on a subject, the application of the microbial MDR inhibitor and/or phenothiazinium and the irradiation with an effective light source can be targeted to that area. For example, wounds, cuts and abrasions in the skin may be targeted by direct application of the microbial MDR inhibitor and/or phenothiazinium to that area. Alternatively, the whole living subject can be treated with the microbial MDR inhibitor and/or phenothiazinium, through, for example, oral or topical administration, followed by irradiation with an effective light source throughout the body.

Administration of the phenothiazinium and the microbial MDR inhibitor can be sequentially (in either order) or at the same time. In one embodiment, the phenothiazinium and the microbial MDR inhibitor can be formulated in the same pharmaceutical composition.

In certain embodiments the phenothiaziniums and/or microbial MDR inhibitors of the present invention are formulated in compositions that also contain one or more additional agents such as pharmaceutically acceptable carriers, excipients, antibiotics, antimicrobial agents (e.g., bactericidal, antiviral or antifungal agents), disinfectants, or detergents. In other embodiments, phenothiaziniums and/or microbial MDR inhibitors of the present invention are co-administered with one or more additional agents such as antibiotics, antimicrobial agents (e.g., bactericidal, antiviral or antifungal agents), disinfectants, or detergents, optionally present within the same composition as the phenothiazinium.

Methods of the present invention further provide a means for sterilizing or decontaminating inanimate objects and substances contain microorganisms.

In one embodiment, food can be decontaminated using methods of the present invention. “Food” includes, but is not limited to, animal-derived products (such as meat, fish, milk, cheese and eggs), plants (such as vegetables, grains, seeds, and oils), plant-derived products, and fungus/fungus-derived products (such as mushrooms, tofu, yeast and yeast-products). The food to be decontaminated can be for consumption by humans or other animals.

In another embodiment, the objects and substances that can be decontaminated using methods of the present invention include but are not limited to animal tissues for transplantation or grafting, products made from human or animal organs or tissues, serum proteins (such as albumin and immunoglobulin), extracellular matrix proteins, gelatin, hormones, bone meal, nutritional supplements, and additionally any material that can be found in a human or animal that is susceptible to infection or that may carry or transmit infection.

In another embodiment “biological fluids” can be decontaminated using methods of the present invention. Biological fluids include but are by no means limited to cerebrospinal fluid, blood, blood products, milk, and semen, and also includes culture medium used for the culture of cells or for the production of recombinant proteins. The term “blood product” includes the red blood cells, white blood cells, serum or plasma separated from the blood. A further aspect of the invention is the use of the claimed methods to treat blood and blood products prior to transfer to a recipient.

In another embodiment, the objects and substances that can be decontaminated using the methods of the present invention are medical instruments, such as catheters, cannulas, dialysis or transfusion devices, shunts, stents, sutures, scissors, needles, stylets, devices for accessing the interior of the body, implantable ports, blades, scalpels. The term “medical instrument” is intended to encompass any type of device or apparatus that is used to contact the interior or exterior of a patient and also includes dental instruments. The term also encompasses any device or tool used in the preparation or manufacture, or otherwise comes into contact with, a biological tissue.

In another embodiment, the objects and substances that can be decontaminated using methods of the present invention are “surfaces.” Surfaces include walls, floors, furniture, any object made of a solid material (such as materials made of wood, metal or plastic), hospital surfaces (such as operating tables) laboratory work surfaces, and food preparation surfaces.

In another embodiment, the objects and substances that can be decontaminated using methods of the present invention include machinery or equipment (such as hospital machinery) and vehicles.

In another embodiment water and air supplies can decontaminated using methods of the present invention. This includes the air and water itself in addition to systems used to deliver air and water such as water tanks, pipes, ventilation ducts and heating/air-conditioning systems.

Microorganisms to be inactivated can be those of any species known in the art that have MDR pumps including but not limited to a bacterium, virus, or fungus, such as any of Staphylococcus (e.g., S. aureus), Streptococcus, Enterococcus, Mycobacterium, Pseudomonas (e.g., P. aeruginosa), Salmonella, Shigella, Escherichia (e.g., E. coli), Erwinia, Klebsiella, Borrelia, Treponema, Campylobacter, Helicobacter, Bordetella, Neisseria, Legionella, Leptospira, Serpulina, Mycoplasma, Bacteroides, Klebsiella, Yersinia, Chlamydia, Vibrio, Actinobacillus, Porphyria, Hemophilus, Helicobacter, Pasteurella, Pseudomonas, Peptostreptococcus, Listeria, Propionibacterium, Mycobacterium, Corynebacterium, Dermatophilus, HIV, Hepatitis virus, Influenza virus, Rhinovirus, Papilloma virus, Measles virus, Herpes virus, Rotavirus, Parvovirus, Psittacosis virus, Ebola virus or Candida (e.g., C. alibcans).

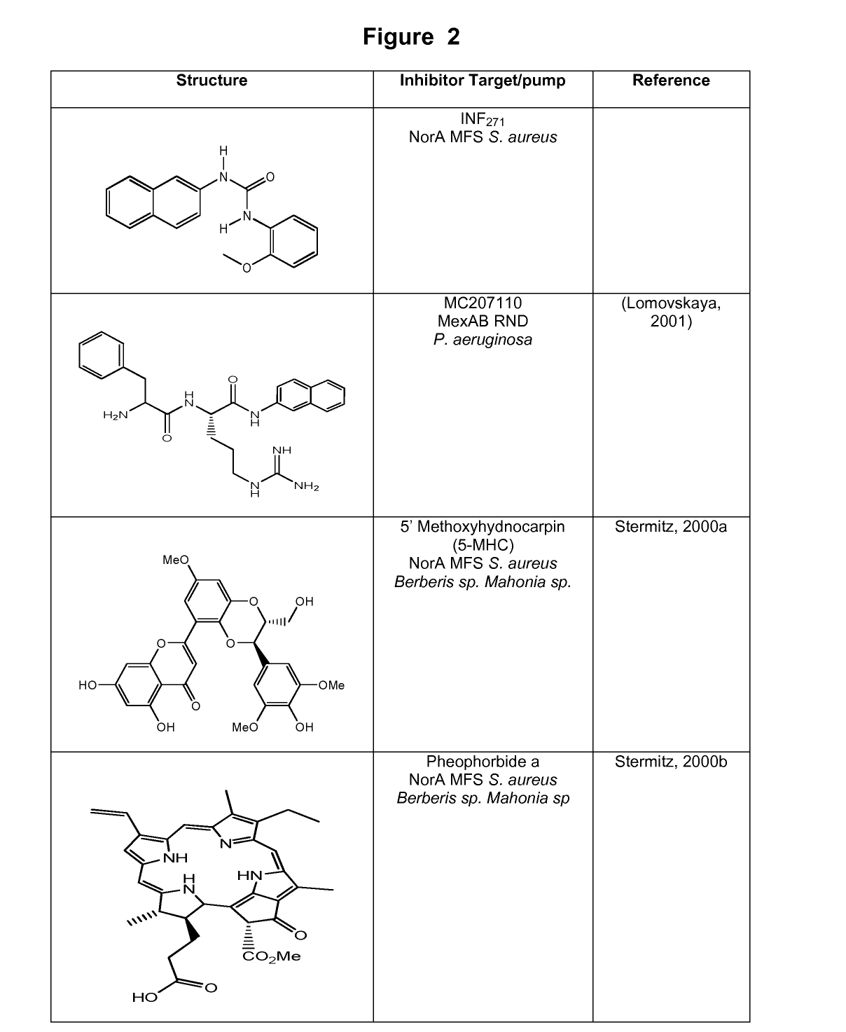

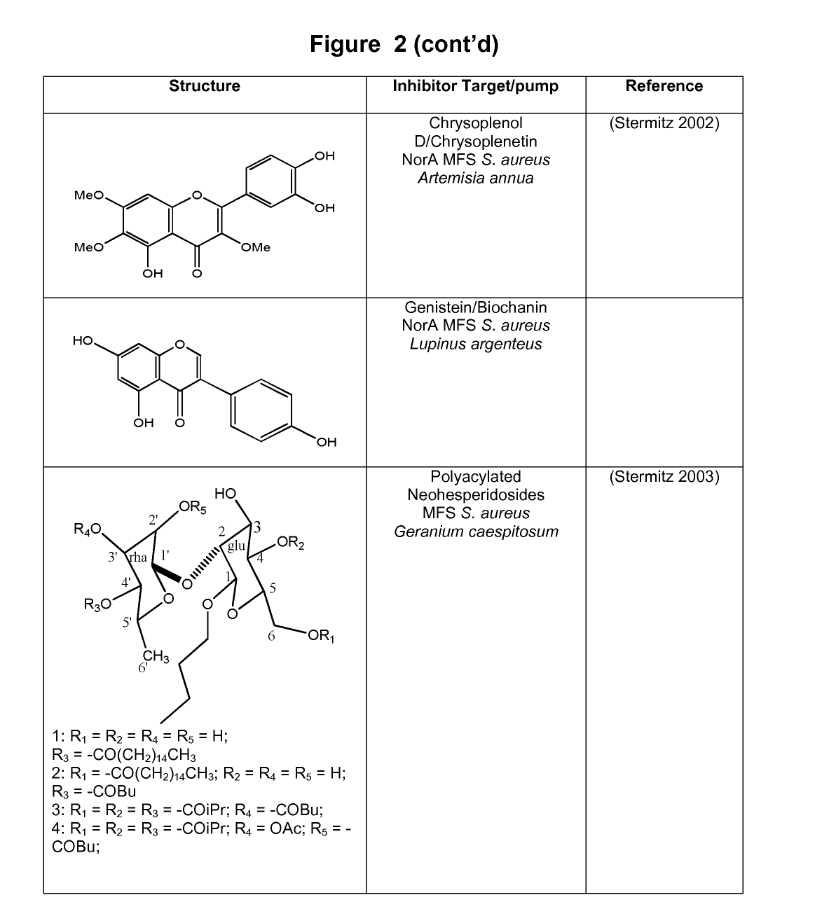

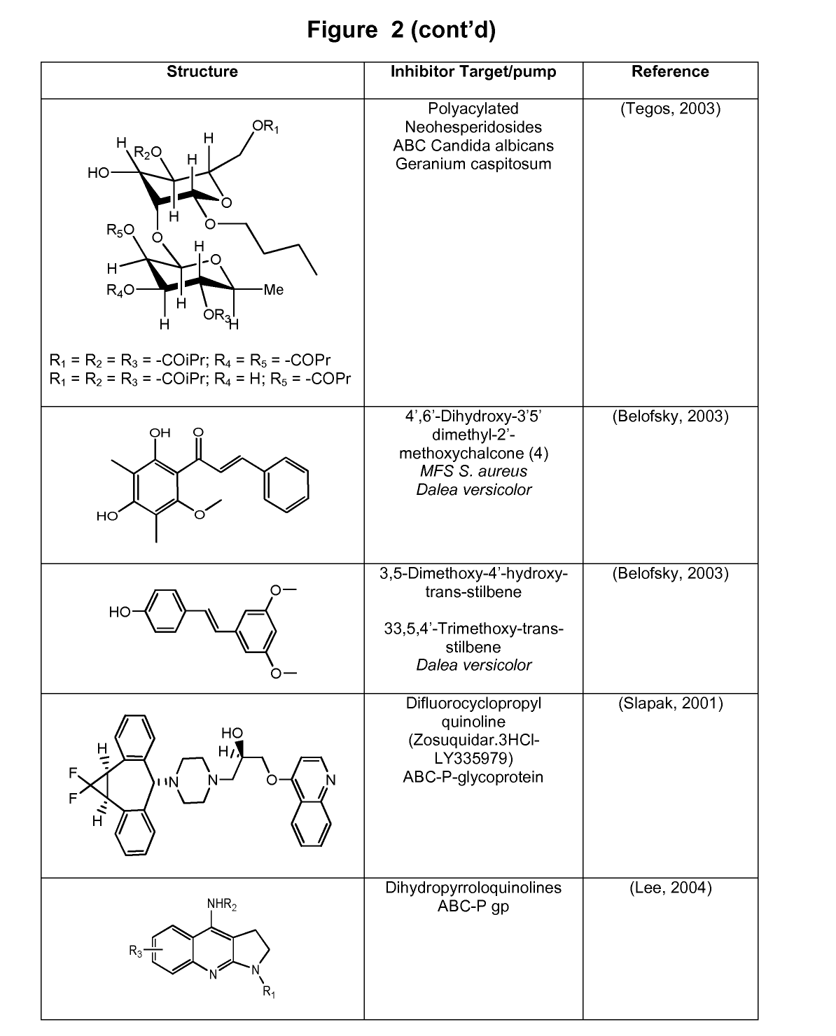

Microbial MDR inhibitors of the invention include but are not limited to INF271 (NorA-Influx Co., Chicago Ill.), MC₂₀₇₁₁₀ (Lomovskaya, 2001, Essential Therapeutics, Mountain View, Calif.), 5′ Methoxyhydnocarpin (“5-MHC”) (Stermitz, 2000a), Pheophorbide a (Stermitz, 2000b), Chrysoplenol D, Chrysoplenetin (Stermitz, 2002), Genistein/Biochanin, Polyacylated Neohesperidosides (Stermitz, 2003), Polyacylated Neohesperidosides (Tegos, 2003), 4′,6′-Dihydroxy-3′,5′ dimethyl-2′-methoxychalcone (Belofsky, 2003), 3,5-Dimethoxy-4′-hydroxy-trans-stilbene, 3,5,4′-Trimethoxy-trans-stilbene (Belofsky, 2003), difluorocyclopropyl quinoline (“Zosuquidar 3HCL or “LY335979”) (Slapak, 2001), Dihydropyrroloquinolines (Lee, 2004), GG918 (Gibbons, 2003), Verpamil Pgp (Rivoltini, 1990), Cyclosporins Pgp, Reserpine Pgp, Propafenone Pgp (Pleban, 2004), pyridazino[4,3-b]indoles Pgp (Velezheva, 2004), Hypericin (Wang, 2004), Cyclooxygenase-2 (Cox-2) (Sorokin, 2004), 3-Oxopiperazinium and Perhydro-3-oxo-1,4-diazepinium derivatives (Masip, 2004), tetrandrine (Liu, 2003), and Phenothiazines (Kolaczkowski, 2003) (FIG. 2).

Phenothiaziniums of the invention include but are not limited to toluidine blue derviatives, toluidine blue O (TBO), methylene blue (MB), new methylene blue N (NMMB), new methylene blue BB, new methylene blue FR, 1,9-dimethylmethylene blue chloride (DMMB), methylene blue derivatives, methylene green, methylene violet Bernthsen, methylene violet 3RAX, Nile blue, Nile blue derivatives, malachite green, Azure blue A, Azure blue B, Azure blue C, safranin O, neutral red, 5-ethylamino-9-diethylaminobenzo[a]phenothiazinium chloride, 5-ethylamino-9-diethylaminobenzo[a]phenoselenazinium chloride, thiopyronine, and thionine.

The phenothiazinium can optionally be linked to a targeting moiety that enhances intracellular localization. In a specific embodiment, the targeting moiety is an antibody. The phenothiazinium can be directly or indirectly linked to an antibody.

Phenothiaziniums of the present invention can optionally be linked to other targeting moieties known in the art, such as peptides that target cell surface receptors, preferably microbial surface receptors. Linkage can be achieved through the use of a coupling agent. The term “coupling agent” as used herein, refers to a reagent capable of coupling a phenothiazinium to a targeting moiety, or to a “backbone” or “bridge” moiety. Any bond which is capable of linking the components such that they are stable under physiological conditions for the time needed for administration and treatment is suitable, but covalent linkages are preferred. The link between two components may be direct, e.g., where a phenothiazinium is linked directly to a targeting moiety, or indirect, e.g., where a phenothiazinium is linked to an intermediate, e.g., linked to a backbone, and that intermediate being linked to the targeting moiety. A coupling agent should function under conditions of temperature, pH, salt, solvent system, and other reactants that substantially retain the chemical stability of the phenothiazinium, the backbone (if present), and the targeting moiety.

A coupling agent can link components without being added to the linked components. Other coupling agents result in the addition of elements of the coupling agent to the linked components. For example, coupling agents can be cross-linking agents that are homo- or hetero-bifunctional, and wherein one or more atomic components of the agent can be retained in the composition. A coupling agent that is not a cross-linking agent can be removed entirely during the coupling reaction, so that the molecular product can be composed entirely of the phenothiazinium, the targeting moiety, and a backbone moiety (if present).

Many coupling agents react with an amine and a carboxylate, to form an amide, or an alcohol and a carboxylate to form an ester. Coupling agents are known in the art, see, e.g., M. Bodansky, “Principles of Peptide Synthesis”, 2nd ed., referenced herein, and T. Greene and P. Wuts, “Protective Groups in Organic Synthesis,” 2nd Ed, 1991, John Wiley, NY. Coupling agents should link component moieties stably, but such that there is only minimal or no denaturation or deactivation of the phenothiazinium or the targeting moiety.

The phenothiazinium compositions of the invention can be prepared by coupling the photosensitizer to targeting moieties using methods described in the following Examples, or by methods known in the art. A variety of coupling agents, including cross-linking agents, can be used for covalent conjugation. Examples of cross-linking agents include N,N′-dicyclohexylcarbodiimide (DCC), N-succinimidyl-5-acetylthioacetate (SATA), N-succinimidyl-3-(2-pyridyldithio)propionate (SPDP), orthophenylenedimaleimide (o-PDM), and sulfosuccinimidyl 4-(N-maleimidomethyl)cyclohexane-1-carboxylate (sulfo-SMCC) (Karpovsky et al. (1984) J. Exp. Med. 160:1686; Liu, M A et al. (1985) Proc. Natl. Acad. Sci, USA 82:8648). Other methods include those described by Paulus and Behring (1985) Ins. Mitt., 78:118-132; Brennan et al. (1985) Science 229:81-83 and Glennie et al., (1987) J. Immunol, 139:2367-2375. A large number of coupling agents for peptides and proteins, along with buffers, solvents, and methods of use, are described in the Pierce Chemical Co. catalog, pages T155-T-200, 1994 (3747 N. Meridian Rd., Rockford Ill., 61105, U.S.A.; Pierce Europe B. V., P. O. Box 1512, 3260 BA Oud Beijerland, The Netherlands), the contents of which are hereby incorporated by reference.

DCC is a useful coupling agent (Pierce #20320; Rockland, Ill.). It promotes coupling of the alcohol NHS in DMSO (Pierce #20684), forming an activated ester which can be cross-linked to polylysine. DCC(N,N′dicyclohexylcarbodiimide) is a carboxy-reactive cross-linker commonly used as a coupling agent in peptide synthesis, and has a molecular weight of 206.32. Another useful cross-linking agent is SPDP (Pierce #21557), a heterobifunctional cross-linker for use with primary amines and sulfhydryl groups. SPDP has a molecular weight of 312.4, a spacer arm length of 6.8 angstroms, is reactive to NHS-esters and pyridyldithio groups, and produces cleavable cross-linking such that, upon further reaction, the agent is eliminated so the phenothiazinium can be linked directly to a backbone or targeting moiety. Other useful conjugating agents are SATA (Pierce #26102) for introduction of blocked SH groups for two-step cross-linking, which is deblocked with hydroxylamine-25-HCl (Pierce #26103), and sulfo-SMCC (Pierce #22322), reactive towards amines and sulfhydryls. Other cross-linking and coupling agents are also available from Pierce Chemical Co. (Rockford, Ill.). Additional compounds and processes, particularly those involving a Schiff base as an intermediate, for conjugation of proteins to other proteins or to other compositions, for example to reporter groups or to chelators for metal ion labeling of a protein, are disclosed in EPO 243,929 A2 (published Nov. 4, 1987).

Phenothiaziniums which contain carboxyl groups can be joined to lysine s-amino groups in the target polypeptides either by preformed reactive esters (such as N-hydroxy succinimide ester) or esters conjugated in situ by a carbodiimide-mediated reaction. The same applies to phenothiaziniums that contain sulfonic acid groups, which can be transformed to sulfonyl chlorides, which react with amino groups. Phenothiaziniums that have carboxyl groups can be joined to amino groups on the polypeptide by an in situ carbodiimide method. Phenothiaziniums can also be attached to hydroxyl groups, of serine or threonine residues or to sulfhydryl groups, of serine or threonine residues or to sulfhydryl groups of cysteine residues.

Methods of joining components of a composition, e.g., coupling polyamino acid chains bearing phenothiaziniums to antibacterial polypeptides, can use heterobifunctional cross linking reagents. These agents bind a functional group in one chain and to a different functional group in the second chain. These functional groups typically are amino, carboxyl, sulfhydryl, and aldehyde. There are many permutations of appropriate moieties that will react with these groups and with differently formulated structures, to join them together (described in the Pierce Catalog and Merrifield et al. (1994) Ciba Found Symp. 186:5-20).

The production and purification of phenothiaziniums coupled to targeting moieties can be practiced by methods known in the art. Yield from coupling reactions can be assessed by spectroscopy of product eluting from a chromatographic fractionation in the final step of purification. Coupling of one or more phenothiazinium molecules to a targeting moiety or to a backbone shifts the peak of absorbance in the elution profile in fractions eluted using sizing gel chromatography, e.g., with the appropriate choice of Sephadex G50, 6100, or 6200 or other such matrices (Pharmacia-Biotech, Piscataway N. J.). Choice of appropriate sizing gel, for example Sephadex gel, can be determined by that gel in which the phenothiazinium elutes in a fraction beyond the excluded volume of material too large to interact with the bead, i.e., the uncoupled starting phenothiazinium interacts to some extent with the fractionation bead and is concomitantly retarded to some extent. The correct useful gel can be predicted from the molecular weight of the uncoupled phenothiazinium. The successful reaction products of phenothiazinium compositions coupled to additional moieties generally have characteristic higher molecular weights, causing them to interact with the chromatographic bead to a lesser extent, and thus appear in fractions eluting earlier than fractions containing the uncoupled phenothiazinium substrate. Unreacted phenothiazinium substrate generally appears in fractions characteristic of the starting material, and the yield from each reaction can thus be assessed both from size of the peak of larger molecular weight material, and the decrease in the peak of characteristic starting material. The area under the peak of the product fractions is converted to the size of the yield using the molar extinction coefficient.

The product can be analyzed using NMR, integrating areas of appropriate product peaks, to determine relative yields with different coupling agents. A red shift in absorption of a phenothiazinium has often been observed following coupling to a polyamino acid. Coupling to a larger carrier such as a protein might produce a comparable shift, as coupling to an antibody resulted in a shift of about 3-5 nm in that direction compared to absorption of the free phenothiazinium.

Phenothiaziniums can be coupled directly to a targeting moiety, such as a scavenger receptor ligand. Other photosensitizer compositions of the invention include a “backbone” or “bridge” moiety, such as a polyamino acid, in which the backbone is coupled both to a phenothiazinium and to a targeting moiety.

Inclusion of a backbone in a composition with a phenothiazinium and a targeting moiety can provide a number of advantages, including the provision of greater stoichiometric ranges of phenothiazinium and targeting moieties coupled per backbone. If the backbone possesses intrinsic affinity for a target organism, the affinity of the composition can be enhanced by coupling to the backbone. The specific range of organisms that can be targeted with one composition can be expanded by coupling two or more different targeting moieties to a single phenothiazinium-backbone composition.

Peptides useful in the methods and compounds of the invention for design and characterization of backbone moieties include poly-amino acids which can be homo- and hetero-polymers of L-, D-, racemic DL- or mixed L- and D-amino acid composition, and which can be of defined or random mixed composition and sequence. These peptides can be modeled after particular natural peptides, and optimized by the technique of phage display and selection for enhanced binding to a chosen target, so that the selected peptide of highest affinity is characterized and then produced synthetically. Further modifications of functional groups can be introduced for purposes, for example, of increased solubility, decreased aggregation, and altered extent of hydrophobicity. Examples of nonpeptide backbones include nucleic acids and derivatives of nucleic acids such as DNA, RNA and peptide nucleic acids; polysaccharides and derivatives such as starch, pectin, chitins, celluloses and hemimethylated celluloses; lipids such as triglyceride derivatives and cerebrosides; synthetic polymers such as polyethylene glycols (PEGS) and PEG star polymers; dextran derivatives, polyvinyl alcohols, N-(2-hydroxypropyl)-methacrylamide copolymers, poly (DL-glycolic acid-lactic acid); and compositions containing elements of any of these classes of compounds.

The affinity of phenothiazinium can be refined by modifying its charge. For example, conjugates including poly-L-lysine can be made in varying sizes and charges (cationic, neutral, and anionic), for example, free NH2 groups of the polylysine are capped with acetyl, succinyl, or other R groups to alter the charge of the final composition. Net charge of a composition of the present invention can be determined by isoelectric focusing (IEF). This technique uses applied voltage to generate a pH gradient in a non-sieving acrylamide or agarose gel by the use of a system of ampholytes (synthetic buffering components). When charged polypeptides are applied to the gel they will migrate either to higher pH or to lower pH regions of the gel according to the position at which they become non-charged and hence unable to move further. This position can be determined by reference to the positions of a series of known IEF marker proteins.

For photoactivation, the wavelength of light is matched to the electronic absorption spectrum of the phenothiazinium so that the phenothiazinium absorbs photons and the desired photochemistry can occur. The wavelength of activating light should be tailored to the absorption band of particular phenothiazinium. In specific embodiments, the activating light is provided at a wavelength of greater than about 400, 500, 600 or 700 nm, or in a range from about 450 nm to about 750 nm.

The effective penetration depth, δ_{eff} of a given wavelength of light is a function of the optical properties of the material being irradiated, such as absorption and scatter. For example, the fluence (light dose) in a tissue is related to the depth, d, as: e^{-d}/δ_{eff}. Typically, the effective penetration depth is about 2 to about 3 mm at 630 nm and increases to about 5 to about 6 nm at longer wavelengths (700-800 nm) (Svaasand and Ellingsen, 1983). In general, phenothiaziniums with longer absorbing wavelengths and higher molar absorption coefficients at these wavelengths are more effective phenothiaziniums.

The effective light dosage will vary depending on various factors, including the amount of the phenothiazinium administered, the wavelength of the photoactivating light, the intensity of the photoactivating light, and the duration of irradiation by the photoactivating light. Thus, the light dose can be adjusted to an effective dose by adjusting one or more of these factors. In general the total fluence applied should be in the range of about 10 to about 1000 J/cm². The determination of suitable wavelength, light intensity, and duration of irradiation is within ordinary skill in the art.

In embodiments where the phenothiazinium is methylene blue (MB), it is preferred that that the irradiating light has a wavelength of about 660 nm and a fluence of up to about 1000 J/cm².

In embodiments where the phenothiazinium is New Methylene Blue (NMB) it is preferred that that the irradiating light has a wavelength of about 635 nm and a fluence of up to about 1000 J/cm².

In embodiments where the phenothiazinium is 1,9-Dimethylmethylene Blue Chloride (DMMB) it is preferred that that the irradiating light has a wavelength of about 660 nm and a fluence of up to about 1000 J/cm².

In embodiments where the phenothiazinium is methylene green it is preferred that that the irradiating light has a wavelength of about 660 nm and a fluence of up to about 1000 J/cm².

In embodiments where the phenothiazinium is methylene violet Bernthsen it is preferred that that the irradiating light has a wavelength of about 600 nm and a fluence of up to about 1000 J/cm².

In embodiments where the phenothiazinium is methylene violet 3RAX it is preferred that that the irradiating light has a wavelength of about 560 nm and a fluence of up to about 1000 J/cm².

In embodiments where the phenothiazinium is malachite green it is preferred that that the irradiating light has a wavelength of about 610 nm and a fluence of up to about 1000 J/cm².

In embodiments where the phenothiazinium is either toluidine blue (TB) or toluidine blue O (TBO) it is preferred that that the irradiating light has a wavelength of about 635 nm and a fluence of up to about 1000 J/cm².

In embodiments where the phenothiazinium is either azure blue A or azure blue B it is preferred that that the irradiating light has a wavelength of about 620 nm and a fluence of up to about 1000 J/cm².

In embodiments where the phenothiazinium is azure blue C it is preferred that that the irradiating light has a wavelength of about 600 nm and a fluence of up to about 1000 J/cm².

In embodiments where the phenothiazinium is neutral red it is preferred that that the irradiating light has a wavelength of about 540 nm and a fluence of up to about 1000 J/cm².

In embodiments where the phenothiazinium is thionine it is preferred that that the irradiating light has a wavelength of about 600-nm and a fluence of up to about 1000 J/cm².

The light for photoactivation can be produced and delivered by any suitable means known in the art. In one embodiment a strong light source such as a searchlight, lamp, light box, laser, light-emitting diode (LED) or optical fiber is used to irradiate the animal or object until the required fluence has been delivered.

In another embodiment natural sunlight is used as light source. Photosensitive dyes are, by definition, light sensitive. Thus, they are totally photobleached and/or degraded following long prolonged exposure to sunlight.

If natural sunlight is used it is preferred, although not essential, that a light meter is used to measure the light dose and dose rate in order that the object or animal is exposed to the sunlight for a sufficient period of time. In some circumstances, such as for decontamination in the field during combat, or for decontamination of large objects or large numbers of people, the use of natural sunlight may be particularly advantageous as it eliminates the need for large numbers of artificial light sources which may be in short supply and may be cumbersome and/or expensive. Furthermore, the use of natural sunlight as the light source is also desirable from an environmental point of view.

An appropriate composition for use in methods of the present invention, which includes a phenothiazinium and/or a microbial MDR inhibitor, can be supplied in various forms and delivered in a variety of ways depending on the specific application. Standard texts, such as Remington: The Science and Practice of Pharmacy, 17^{th} edition, Mack Publishing Company, incorporated herein by reference, can be consulted to prepare suitable compositions and formulations for administration, without undue experimentation.

Compositions of the present invention are administered by a mode appropriate for the form of the composition and the tissue/site to be treated. Compositions can be supplied in solid, semi-solid or liquid forms, including tablets, capsules, powders, liquids, lotions, creams, suspensions, spays and aerosols.

In one embodiment, the compositions are administered topically to the skin, or in particular to cuts, abrasions or other wounds in the skin. In this case, suitable forms for administration of the composition include creams, lotions, washes, and sprays. Other routes of topical administration may include application to the hair or eyes. In the case of application to the eyes, a bathing solution or eye drops are a preferred form of delivery.

In one embodiment, the compositions of the present invention comprise a simple aqueous solution containing an effective amount of the desired phenothiazinium and/or microbial MDR inhibitor in sterile water, phosphate buffered saline, or some other aqueous solvent. Additionally such aqueous solutions may also contain pH buffering agents and preservatives and antimicrobial agents. Typically the amount of the phenothiazinium present in such an aqueous solution formulation is in the range of about 0.0001% to about 50% weight/volume, or the phenothiazinium may be present at concentrations ranging from about 0.1 μM to about 100 mM.

Such aqueous solution formulations are well suited to applications where bathing solutions, such as soaks or eye drops, or sprays are required. The aqueous solutions can be administered to a specific site on a living animal or may be used to bathe or douse the whole animal. For example, in one embodiment the compositions of the present invention may be animal or human “dips”.

Thus, in one embodiment an aqueous solution containing the desired phenothiazinium and/or microbial MDR inhibitor is used to soak or spray an affected part of the body, such as, for example, the eyes, and then either at the same time or after bathing, the affected part of the body is irradiated with an effective source of light.

In other embodiments, the compositions can be applied topically in the form of creams, lotions, ointments and the like. Many formulations of suitable “base” creams and lotions for topical application are known in the art, and any such formulation can be used. By “base” is meant the formulation of the composition without the actual active substance. For example, in the case of an antibiotic cream, the “base” is all of the components of the cream other than the antibiotic. An effective amount of the chosen phenothiazinium can be added to the “base” cream and lotion formulations as taught by U.S. Pat. Nos. 6,621,574, 5,874,098, 5,698,589, 5,153,230 and 6,607,753. The chosen phenothiazinium and/or microbial MDR inhibitor can be mixed with any known “base” cream, ointment or lotion known in the art to be safe for topical application. In some embodiments, other active agents may be added to the phenothiazinium composition, such as antibiotics or antimicrobial agents. It is envisaged that the final concentration of phenothiazinium and/or microbial MDR inhibitor in the cream, lotion or ointment will be between about 0.0001% and about 50% of the final composition, depending upon factors such as the specific phenothiazinium used.

Suitable compositions for the “base” of the creams, lotions, and ointments of the present invention comprise a solvent (such as water or alcohol), and an emollient (such as a hydrocarbon oil, wax, silicone oil, vegetable, animal or marine fat or oil, glyceride derivative, fatty acid or fatty acid ester, alcohol or alcohol ether, lecithin, lanolin and derivatives, polyhydric alcohol or ester, wax ester, sterol, phospholipid and the like), and generally also contain an emulsifier (nonionic, cationic or anionic), although some emollients inherently possess emulsifying properties and thus in these situations an additional emulsifier is not necessary. These “base” ingredients can be formulated into either a cream, a lotion, a gel, or a solid stick by utilization of different proportions of the ingredients and/or by inclusion of thickening agents such as gums, hydroxypropylmethylcellulose, or other forms of hydrophilic colloids.

In one embodiment, such phenothiazinium and/or microbial MDR inhibitor-containing creams, ointments and lotions are applied topically to the skin, mucous membranes (such as the oral cavity) or hair and then irradiated with the effective light source.

An alternative means of treatment is to produce compositions in dry powdered form that can be inhaled. Where delivery by inhalation is desired, as much as possible of the phenothiazinium powder of the present invention should consist of particles having a diameter of less than about 10 microns, for example about 0.01 to about 10 microns or about 0.1 to about 6 microns, for example about 0.1 to about 5 microns, or agglomerates of said particles. Preferably at least 50% of the powder consists of particles within the desired size range. These powders need not contain other ingredients. However compositions containing the phenothiazinium and/or microbial MDR inhibitor of the present invention may also include other pharmaceutically acceptable additives such as pharmaceutically acceptable adjuvents, diluents and carriers. Carriers are preferably hydrophilic such as lactose monohydrate. Other suitable carriers include glucose, fructose, galactose, trehalose, sucrose, maltose, raffinose, maltitol, melezitose, stachyose, lactitol, palatinite, starch, xylitol, mannitol, myoinositol, and the like, and hydrates thereof, and amino acids, for example alanine, and betaine.

Administration to the respiratory tract may be effected for example using a dry powder inhaler or a pressurised aerosol inhaler. Suitable dry powder inhalers include dose inhalers, for example the single dose inhaler known by the trade mark Monohaler™ and multi-dose inhalers, for example a multi-dose, breath-actuated dry powder inhaler such as the inhaler known by the trade mark Turbuhaler™.

In other embodiments, compositions of the present invention are formulated for delivery by injection. In one embodiment a sterile solution the desired phenothiazinium in an aqueous solvent (e.g. phosphate buffered saline) is administered be injection intradermally, subcutaneously, intramuscularly or, intravenously.

In other embodiments, compositions for injection also preferably include conventional pharmaceutically acceptable carriers and excipients which are known to those of skill in the art. Many different “base” formulations are known in the art to be suitable for preparation and delivery of active agents by injection, and any of these can be used. For example, suitable injectable“base” compositions are taught by U.S. Pat. No. 6,326,406.

Injectable compositions can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like. In addition, if desired, the injectable compositions to be administered may also contain minor amounts of non-toxic auxiliary substances such as wetting or emulsifying agents, pH buffering agents and the like, such as for example, sodium acetate, sorbitan monolaurate, triethanolamine oleate.

For example a formulation comprising a sterile solution of the desired phenothiazinium and/or microbial MDR inhibitor at a concentration of about 1 μM to about 100 mM in physiological saline solution is injected intradermally, subcutaneously, intramuscularly, or intravenously. Treatment is then completed by irradiating the affected individual, or a specific site on that individual such as the injection site, with an effective light source, either at the time of, or following, the injection of the composition. In one embodiment the composition is injected in the vicinity of a region of the body that is believed to be contaminated with microorganisms, such as a scratch, abrasions, cut or other wound in the skin. In other embodiments the composition may be delivered systemically, for example, by intravenous injection.

Another suitable method for administration of compositions of the present invention is to implant a slow-release or sustained-release system, such that a constant level of dosage is maintained. See, e.g., U.S. Pat. No. 3,710,795, which is incorporated herein by reference. Compositions may also be administered by transdermal patch (e.g., iontophoretic transfer) for local or systemic application. In both cases, the site of the implant or patch is irradiated with an effective light source to complete the treatment.

Any such treatments described herein may be performed only once, or as frequently as desired until the microorganisms are inactivated. For example, successive administrations at hourly intervals can be used. Alternatively, treatment may be performed twice daily or as directed by a physician.

The present invention is additionally described by way of the following illustrative, non-limiting examples, which provide a better understanding of the present invention and its many advantages.

### EXAMPLES

The following experimental conditions were employed:

### Microbial Strains and Culture Conditions.

Bacterial strains used throughout the following Examples are listed in Table 1, below.

TABLE 1

| | | Source/ |
|---|---|---|
| Bacterial Strain | Genotype | Reference |
| Staphylococcus aureus 8325-4 | WT | (Kaatz, 2000) |
| S. aureus 1758 | S. aureus norA: Cm | K. Lewis |
| S. aureus | S. aureus norA | D. C. Hooper |
| Escherichia coli K-12 | WT | WT |
| E. coli KLE701 | E. coli tolC::tet | K. Lewis |
| Pseudomonas aeruginosa PA767 | WT | K. Lewis |
| P. aeruginosa K1119 | PAO1 Δ mexAB-oprM | (Li, 1998) |
| P. aeruginosa | PAO1 Δ mexAB-oprM | (Li, 1998) |

Cells were cultured in brain-heart infusion (BHI) broth with aeration at 37° C. Cells were used for experiments in mid-log growth phase (10⁸ per mL).

### Photosensitizers and Light Sources.

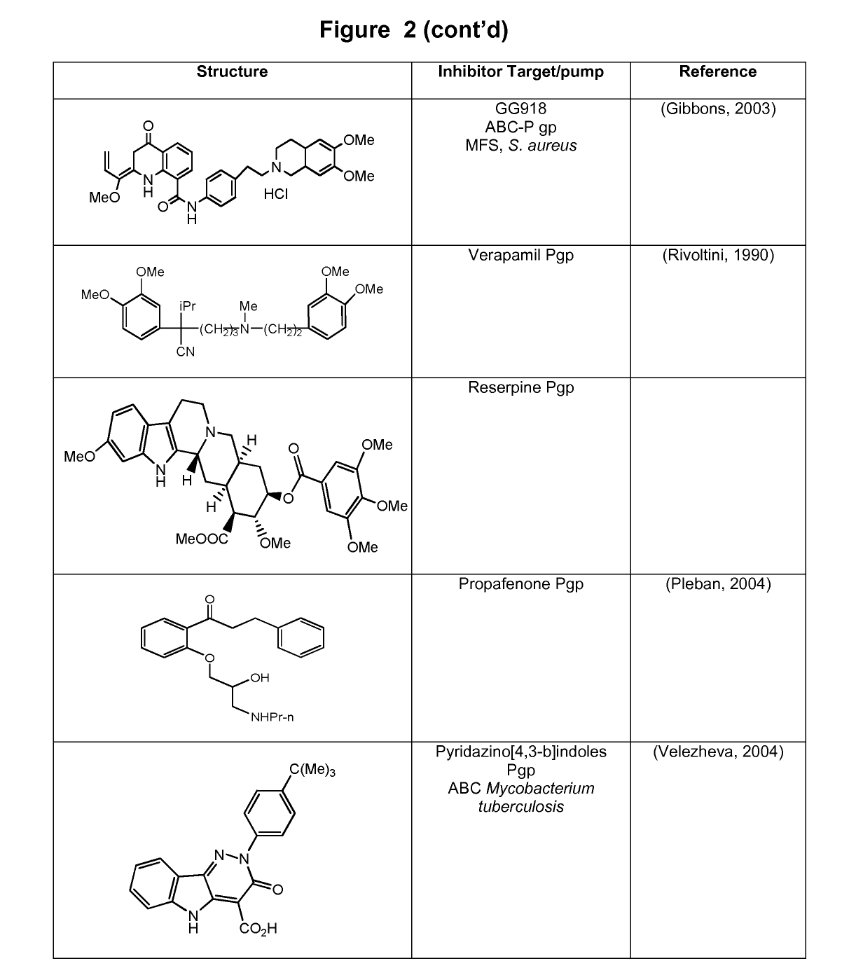

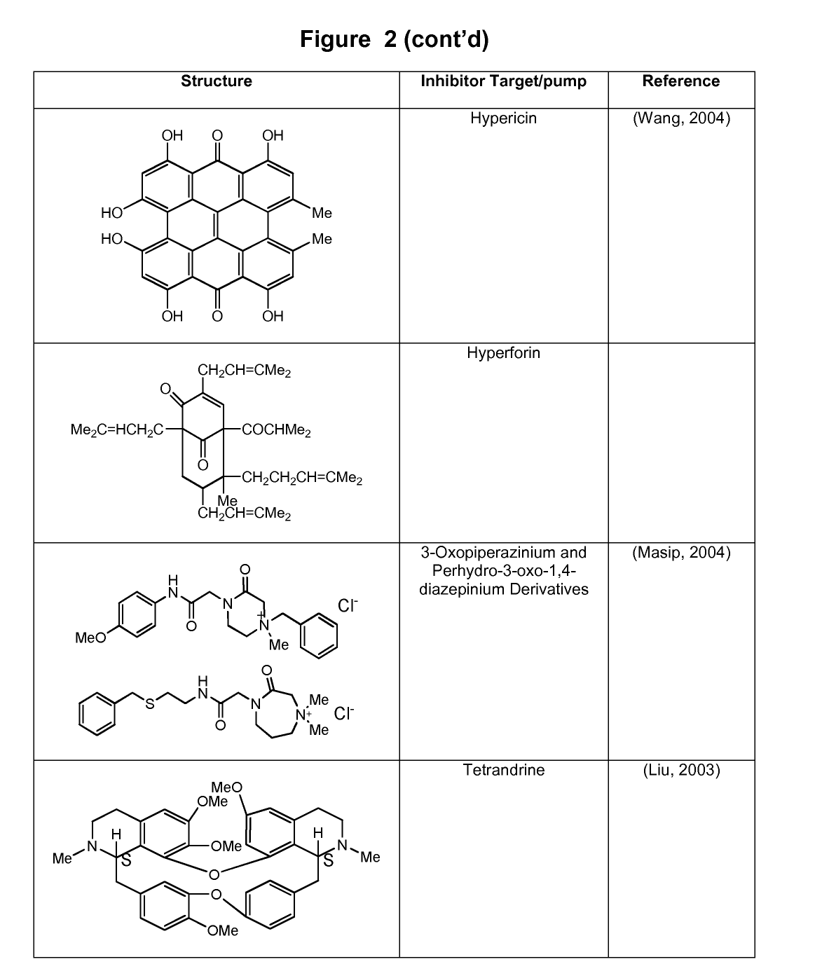

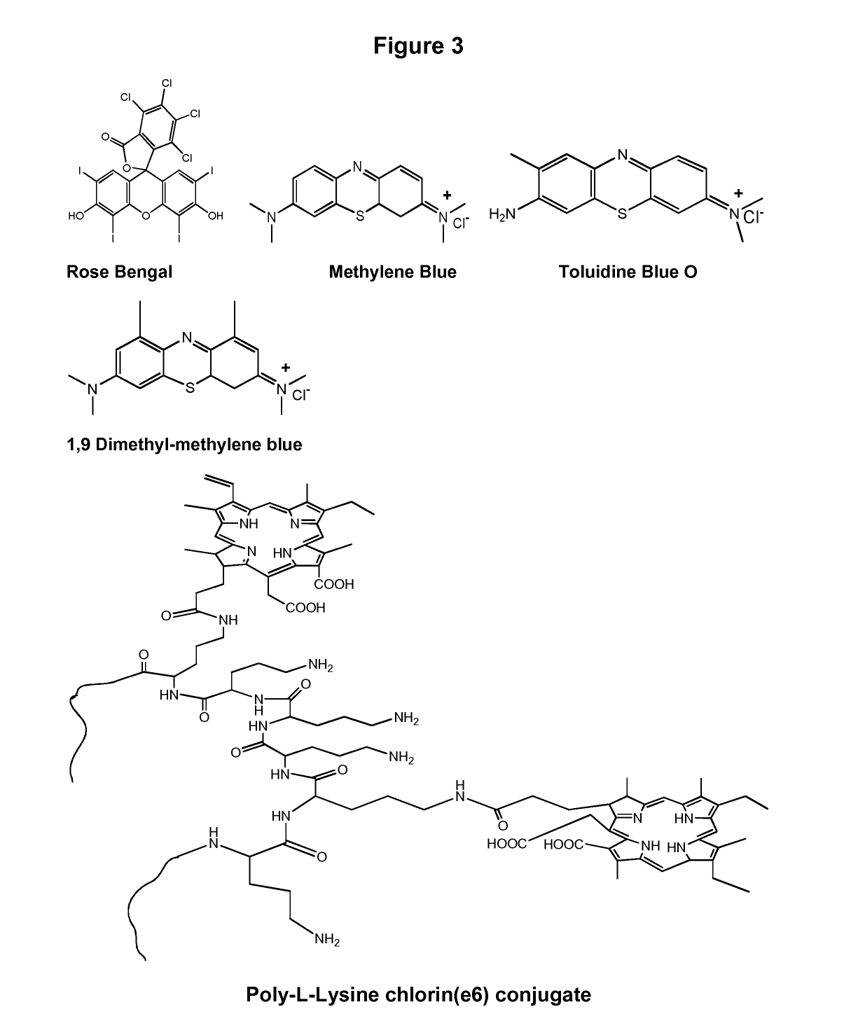

Toluidine blue O (TBO), methylene blue (MB), and 1,9-dimethylmethylene blue (DMMB), all as chloride salts (Sigma-Aldrich St. Louis, Mo.), were used as phenothiazinium-based photosensitizer. A polylysine-chlorinₑ₆ conjugate (pL-cₑ₆), and Rose Bengal (RB) (Sigma-Aldrich) were used as non-phenothiazinium-based PS (FIG. 3). Stock solutions were prepared in water at a concentration of 2-mM and stored for a maximum of 2 weeks at 4° C. in the dark before use. Spectra of stock solutions diluted 140-280 fold in methanol were recorded on an UV-visible spectroscopy system (Waldbronn, Germany). A non-coherent light source with interchangeable fiber bundles (LC122, LumaCare, London, UK) was employed. Thirty-nm band pass filters at ranges 540±15 nm for RB, 635±15 nm for TBO, and DMMB, and 660±15 nm for MB and pLce6 were used. The total power output provided out of the fiber bundle ranged from 300-700 mW. The spot was arranged to give an irradiance of 100 mW/cm².

### Photodynamic Inactivation (PDI) Studies.

Bacterial suspensions in PBS (initial concentration 10⁸ cells mL⁻¹) were incubated with PS in the dark at room temperature for 30 minutes at concentrations varying from 1-μM to 300-μM. The cell suspensions were centrifuged at 12000 rpm and then washed twice with sterile PBS. The bacterial suspensions were placed in wells of 96 well micro titer plates (Fisher Scientific) and illuminated with appropriate light at room temperature. Fluences ranged from 0 to 20 Jcm⁻² at a fluence rate of 100 mWcm⁻². During illumination, aliquots of 10 were taken to determine the colony-forming units. The contents of the wells were mixed before sampling. The aliquots were serially diluted 10-fold in PBS to give dilutions of 10⁻¹-10⁻⁶ times the original concentrations and were streaked horizontally on square BHI agar plates as described by Jett et al (1997). This allowed a maximum of seven logs of killing to be measured. Plates were incubated at 37° C. overnight. Two types of control conditions were used: illumination in the absence of photosensitizer and incubation with photosensitizer in the dark.

### Uptake Studies.

Bacteria suspensions (10⁸ cells/mL) were incubated in PBS in the dark at room temperature for 30 min with photosensitizer in the same concentrations as were used for the PDI experiments. Incubations were carried out in triplicate. The cell suspensions were centrifuged (9,000×g, 1 min), the photosensitizer solution was aspirated, and bacteria were washed twice in 1 mL of sterile PBS and centrifuged as described above. Finally, the cell pellet was dissolved by digesting it in 3 mL of 0.1 M NaOH-1% sodium dodecyl sulfate (SDS) for at least 24 h to give the cell extract as a homogenous solution. Fluorescence in the extracts was measured on a spectrofluorimeter (model FluoroMax3; SPEX Industries, Edison, N. J.).

For TBO and DMMB, the excitation wavelength was 620 nm, and the range for emission was 627 to 720 nm. For MB, the excitation wavelength was 650 mm, and the range for emission was 655 to 720 nm. For pL-ce6, the excitation wavelength was 400 nm, and the emission spectra of the solution were recorded from 580 to 700 nm. For RB, the excitation wavelength was 552 nm, and the emission was recorded in the range from 555 to 620 nm. The fluorescence was calculated form the height of the peaks recorded. If necessary, the solution was diluted with 0.1 M NaOH-1% SDS to reach a concentration of the photosensitizer where the fluorescence response was linear. Calibration curves were made from pure photosensitizer dissolved in NaOH/SDS and used for the determination of photosensitizer concentration in the suspension. Uptake values were obtained by dividing the number of nmol of PS in the dissolved pellet by the number of CFU obtained by serial dilutions and the number of PS molecules/cell calculated by using Avogadro's number.

### Statistics.

Values are means of three separate experiments and bars are SEM. Differences between means were tested for significance by an unpaired two-tailed Students t-test assuming equal or unequal variations as appropriate. The significance level was set at p<0.05.

### Example 1

### Phenothiaziniums are Designated Substrates of Microbial MDRs

NorA Expression Protects Against MB Phototoxicity in S. aureus.

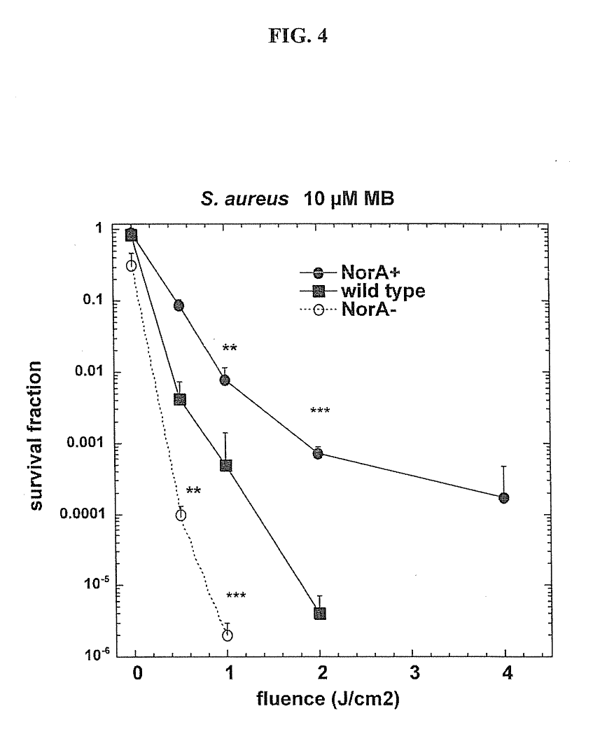

The three isogenic strains of S. aureus were incubated with 10 μM MB for 30 minutes and then washed free of unbound dye by centrifugation and resuspension in PBS and illuminated with 100 mWcm⁻² 660-nm light, and the survival fractions determined as described above. FIG. 4 shows the resulting light-dose dependent phototoxicity. The wild-type strain showed 3 logs of killing after 1 J/cm², 5 logs after 2 J/cm² and 7 logs after 4 J/cm². The NorA knock-out showed complete killing after 1 J/cm², while the NorA overexpressing strain was significantly protected compared to wild-type (1 log less killing at 1 J/cm², 2 logs less killing at 2 J/cm², and 3 logs less killing at 4 J/cm².

E. coli TolC Knock-Out Mutant is More Susceptible to MB-PDI.

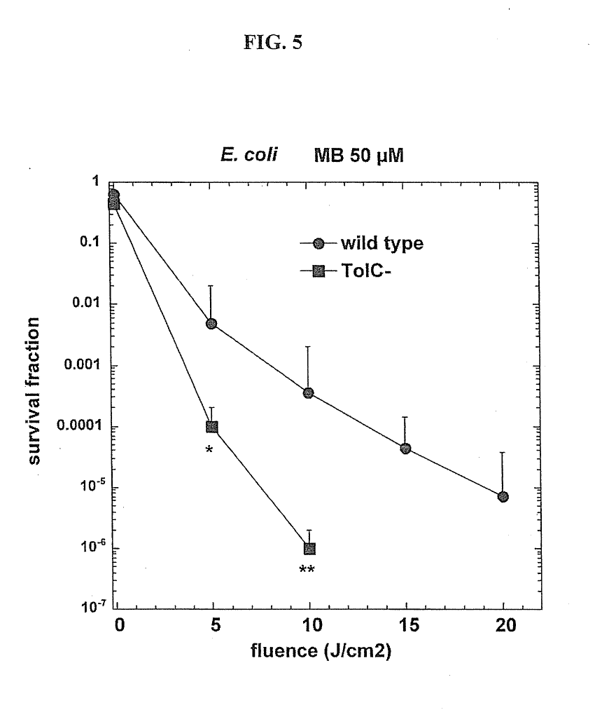

It was necessary to use higher overall PDI doses to kill the Gram-negative E. coli compared to the Gram-positive S. aureus. A concentration of MB of 50 μM was selected with the same 30-minute incubation and wash by centrifugation, together with light doses up to 20 J/cm². Under these conditions, as shown in FIG. 5, the TolC knock-out mutant showed 2 logs more killing than wild-type at 5 J/cm², and three logs more at 10 J/cm², with the knock-out being totally eliminated at higher light doses. When the MB concentration was raised to 250 μM, both the wild-type and TolC knock-out strains were totally eliminated after 20 J/cm² (data not shown).

P. aeruginosa MexAB Expression Determines Phototoxicity of MB-PDI.

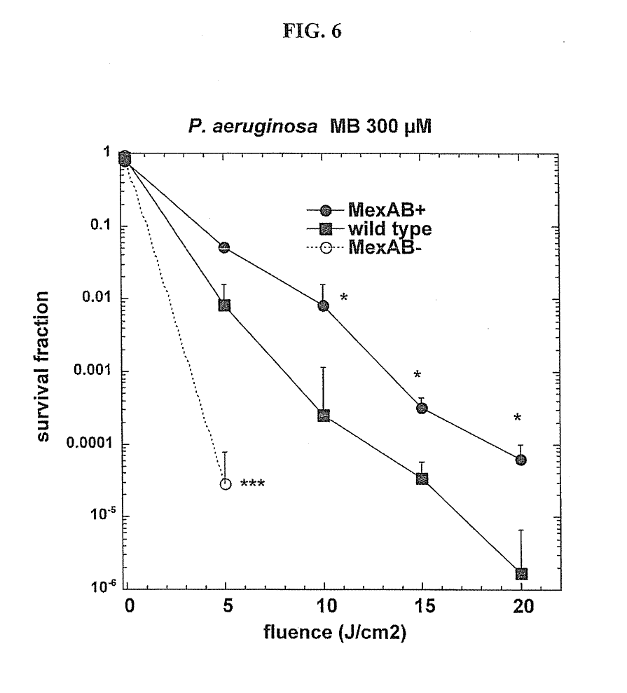

It was necessary to use even higher concentrations of MB than used for E. coli in order to effect light-dependent killing of P. aeruginosa. The three isogenic strains were, therefore, incubated with 300 μM under the same conditions used previously. FIG. 6 shows that the wild-type strain showed 5 logs of killing after 20 J/cm2. The MexAB knock-out showed two logs more killing at 5 and 10 J/cm2 and was completely eliminated at 15 J/cm2. The MexAB overexpressing strain was protected at all light doses by about 1 log.

Multiple Phenothiazinium Photosensitizers are Substrates of S. aureus NorA MDR.

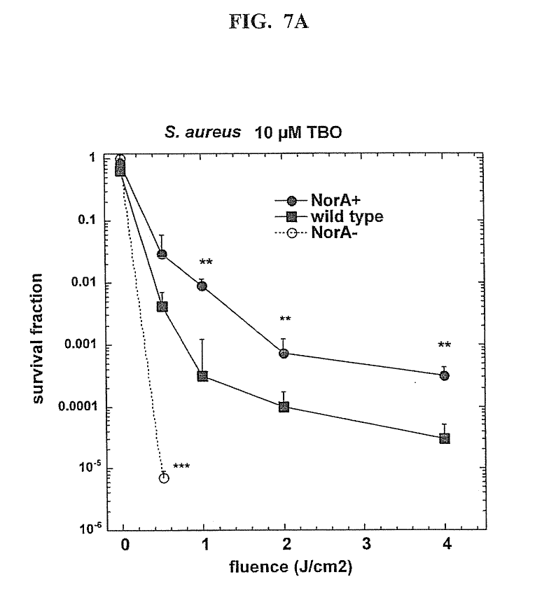

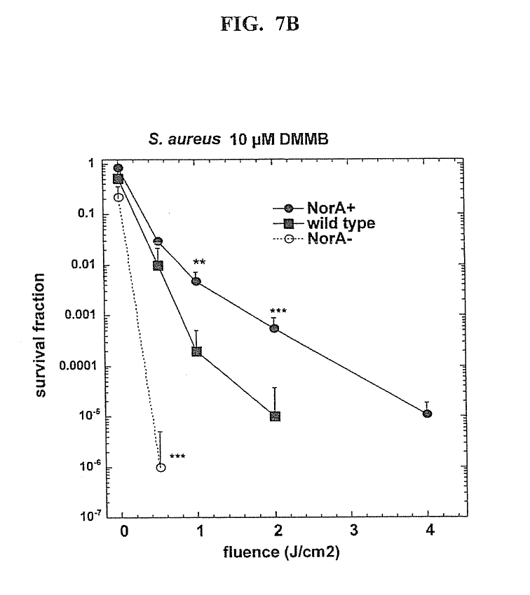

To establish the recognition of phenothiazinium dyes as a class by NorA, the experiments described in FIG. 4 were repeated with the phenothiazinium compounds TBO and DMMB. Incubation with the photosensitizer was for 30 min followed by a wash. Bacteria were then illuminated with 100 mWcm⁻² 635-nm light for both TBO and DMMB. The results shown in FIGS. 7a and 7b show a similar pattern to the susceptibilities as was found for MB. The NorA knockout strain is eliminated by 1 J/cm2 in the case of TBO and by 0.5 J/cm2 in the case of DMMB. By contrast, the wild type strain is comparatively resistant demonstrating 2-4 logs less killing. The NorA overexpression strain shows even less killing than wild-type (roughly 2 logs), and these differences are significant. The overall order of efficiency of killing was DMMB>TBO>MB.

### Activity of Non-Phenothiazinium Photosensitizers is Unaffected by MDR Phenotype.

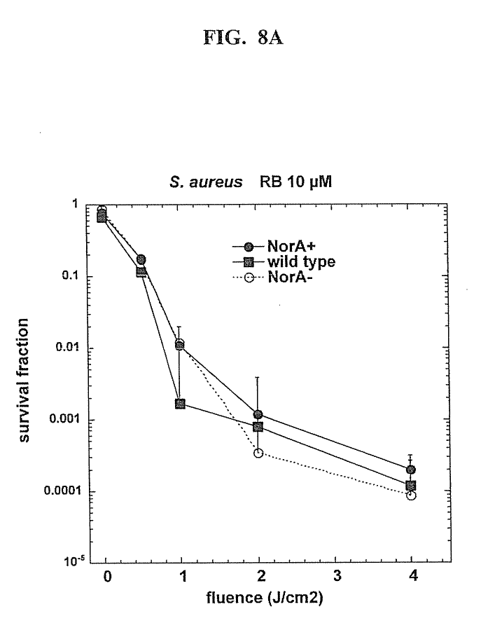

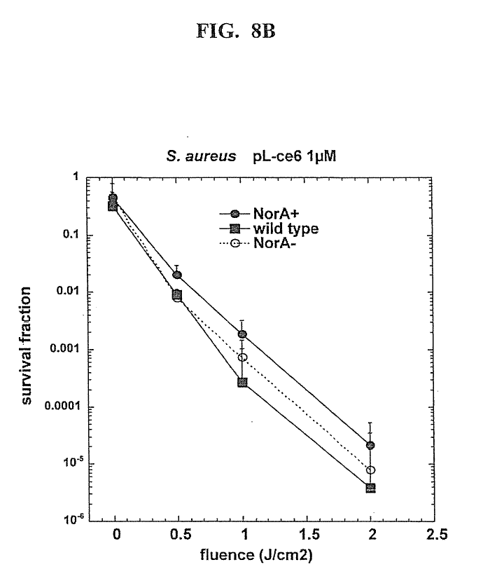

In order to show that the differences in killing observed with the various MDR phenotypes were dependent on MDR recognition of phenothiazium dyes rather than some alternative alteration in microbial physiology that could potentially alter susceptibility to PDI, two antimicrobial photosensitizers with non-phenothiazinium-based molecular structures were studied. Rose Bengal (RB) is a xanthene dye that has four aromatic rings, but these are positioned differently to phenothiazinium dyes, and, in addition, RB possesses an overall negative charge. pL-ce6 is a macromolecular conjugate between the tetrapyrrole photosensitizer chlorin(e6) and a poly-L-lysine chain with an overall polycationic charge that is thought to be taken up by bacteria by disturbing their membrane structure. As seen in FIGS. 8a and 8b, there were no differences in killing between the three S. aureus NorA phenotypes with either photosensitizer. pL-ce6 was significantly more effective than RB since only one tenth the concentration produced more killing with the same light fluence.

### MDR Phenotype Affects Bacterial Uptake of Phenothiazinium Photosensitizers, but not Other Structures.

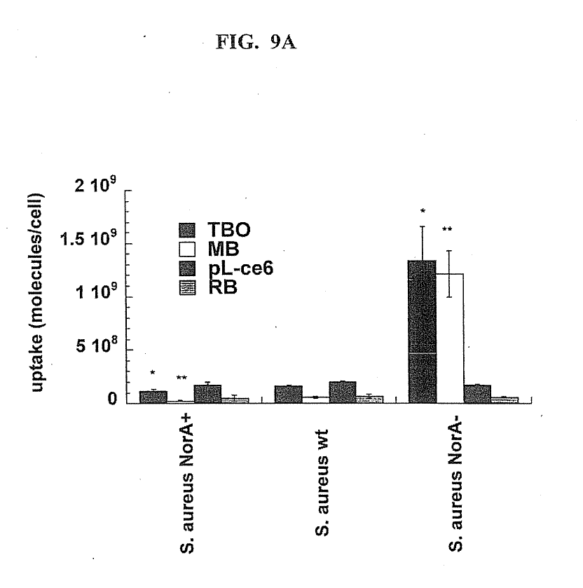

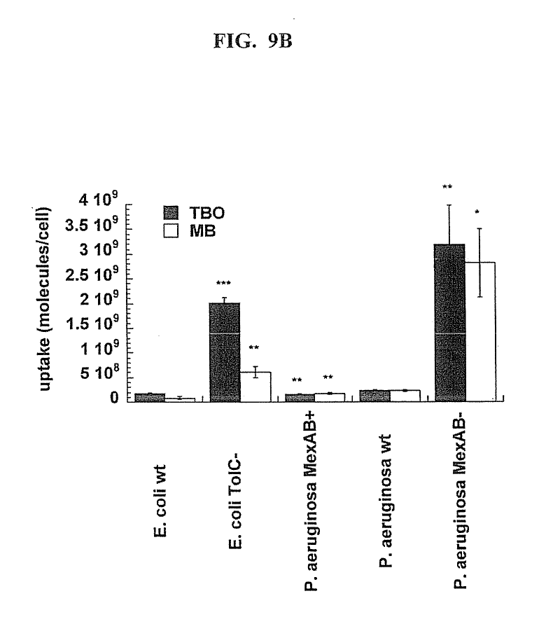

To confirm the hypothesis that the MDR pumps reduce intracellular concentrations of phenothiazinium photosensitizer by an active efflux mechanism, uptake of the dye by the cells was measured by extraction and fluorescence quantification. The cells were incubated with same concentrations of the dye that were used for the killing experiments. Concentrations were 10 μM for MB, TBO, RB and 1 μM for pL-ce6. Photosensitizers were incubated for 10 min, washed, and fluorescence extracted and measured as described. FIG. 9a shows that the uptake of the two phenothiazinium dyes tested (MB and TBO both at 10 μM) by the three S. aureus strains were significantly different according to NorA phenotype. NorA− took up 1.34±0.32×10⁹ and 1.22±0.22×10⁹ molecules/cell of TBO and MB respectively, compared to 0.16±0.02×10⁹ and 0.06±0.01×10⁹ for wild-type, and 0.114±0.016×10⁹ and 0.021±0.003×10⁹ for NorA+. All these differences were significant.

By contrast, the uptakes of the non-phenothiazinium dyes RB (10 μM) and pL-ce6 (1 μM) showed no significant differences between NorA phenotypes. FIG. 9b depicts the uptakes of two phenothiazinium photosensitizers (MB and TBO) by the E. coli wild type and TolC null cells (concentration used was 50 μM), and by the three MexAB phenotypes of P. aeruginosa (concentration used was 300 μM). Of note, the uptakes of the phenothiazinium dyes by all the MDR knockout mutants of different bacterial species is fairly similar (1.2-3.2×10⁹ molecules per cell). In all cases, TBO uptake was higher than MB uptake. This similarity in uptakes between different bacteria is remarkable because of the widely different photosensitizer concentrations used (10-300 μM). These values indicate that there is a necessary amount of photosensitizer per cell to mediate efficient PDI and shows that bacterial uptake of phenothiazinium dyes varies between bacterial classes and species. This variation may be due to intrinsic permeability differences or to the fact that some species (such as P. aeruginosa) may have many separate but related MDR pumps, and knocking out MexAB may still leave other functional MDRs to pumps out phenothiazinium dyes.

### Example 2

### MDR Inhibitors Potentiate the Photodestructive Efficiency of Phenothiaziniums

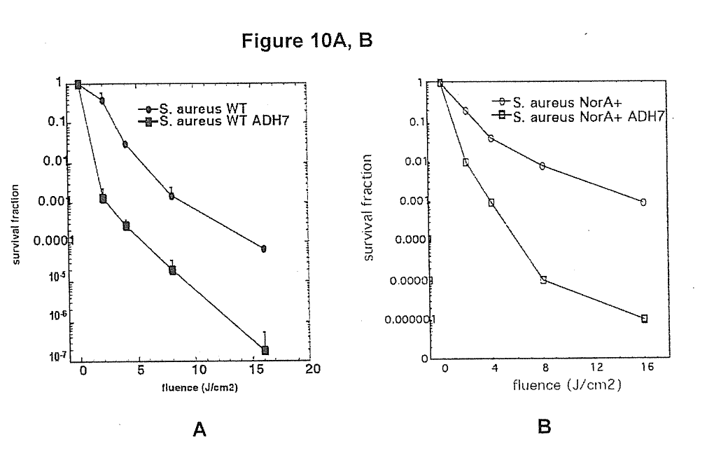

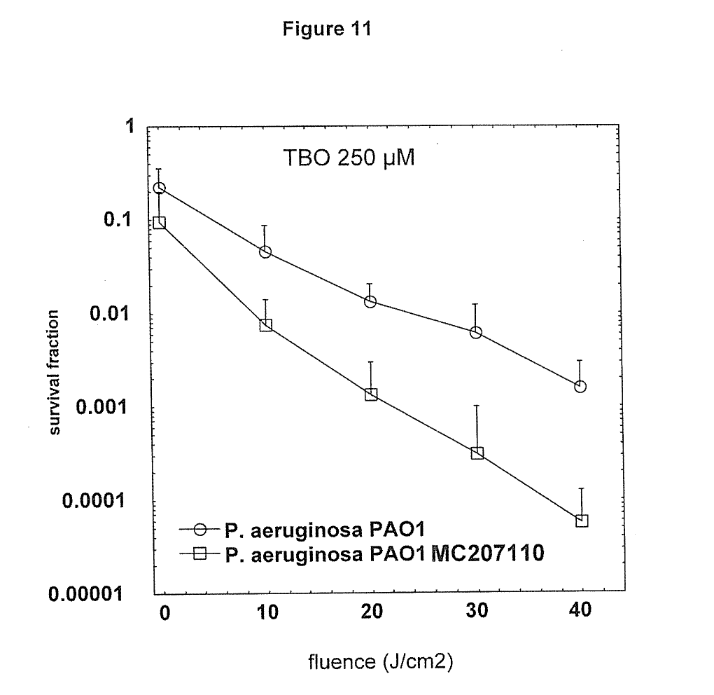

MDR Inhibitors were tested for the ability to potentiate the action of phenothiaziniums. MDR inhibitors were used in a final concentration of 10 ug/ml. Incubation with the photosensitizer was for 30 minutes followed by a wash. Bacteria were then illuminated with 100 mWcm⁻² 635-nm light for Methylene Blue (MB). The neohesperidoside ADH7 was efficient against wild type and overexpression S. aureus resulting in 7 and 5 logs of killing at 8 Jcm⁻² (FIGS. 10a,b), whereas MC207110 potentiated the action of Toluidine Blue O (TBO), resulting in 5 logs of killing in P. aeruginosa (FIG. 11). For the latter, incubation with the photosensitizer was for 30 minutes followed by a wash. Bacteria were then illuminated with 100 mWcm⁻² 660-nm light and the survival fractions determined as described above. These results demonstrate that MDR inhibitors potentiate the photodestructive efficiency of phenothiaziniums

### REFERENCES

Agarwal, S. K., Singh, S. S., S. Verma, & S. Kumar (2000) J Ethnopharmacol 72, 43-46.
Athar, M., Mukhtar, H. & Bickers, D. R. (1988) J Invest Dermatol. 90, 652-657.
Barabote, R. D., Johnson, O. L., Zetina, E., San Francisco, S. K., Fralick, J. A., San Francisco, M. J. (2003) J Bacteriol 185, 5772-5778.
Beck, W., Cirtain, M., Glover, C., Felsted, R., and A. Safa (1988) Biochem. Biophys. Res. Com 153, 959-966.
Bellin, J. S., Lutwick, L. & Jonas, B. (1969) Arch Biochem Biophys 132, 157-164.
Belofsky, G., Percivill, D., Lewis, K., Tegos, G.,& J. Ekart (2003) J Natural Products in press.
Bertoloni, G., Rossi, F., Valduga, G., Jori, G. & van Lier, J. (1990) FEMS Microbiol. Lett. 59, 149-155.
Bezman, S. A., Burtis, P. A., Izod, T. P. & Thayer, M. A. (1978) Photochem Photobiol 28, 325-329.
Bhatti, M., MacRobert, A., Meghji, S., Henderson, B. & Wilson, M. (1998) Photochem Photobiol. 68, 370-376.
Boehncke, W. H., Elshorst-Schmidt, T. & Kaufmann, R. (2000) Arch Dermatol 136, 271-272.
Booth, J. C., & Stern, H. (1972) J Med Microbiol 5, 515-528.
Boyle, R. W., & Dolphin, D. (1996) Photochem. Photobiol. 64, 469-485.
Brendel, M. (1973) Mol Gen Genet 120, 171-180.
Bressler, N. M., & Bressler, S. B. (2000) Invest Ophthalmol Vis Sci, 41, 624-628.
Chung, Y. J., & Saier, M. H. Jr. (2001) Curr Opin Drug Discov Devel 4, 237-245.
Cooney, J. J., & Krinsky, N. I. (1972) Photochem Photobiol 16, 523-526.
Cowan, M. (1999) Clin Microbiol Rev 4, 564-582.
Dahl, T. A., Midden, W. R. & Hartman, P. E (1987) Photochem Photobiol 46, 345-352.
Dahl, T. A., Midden, W. R. & Hartman, P. E. (1989) J Bacteriol 171, 2188-2194.
de Mol, N. J., Beijersbergen van Henegouwen, G. M., Mohn, G. R., Glickman, B. W. & van Kleef, P. M. (1981) Mutat Res 82, 23-30.
Didry, N., L. Dubreuil, and M. Pinkas (1994) Pharmazie 49, 681-683.
Dixon, R. (2001) Nature 411, 843-847.
Dougherty, T. J., Gomer, C. J., Henderson, B. W., Jon, G., Kessel, D., Korbelik, M., Moan, J. & Peng, Q. (1998) J Natl Cancer Inst 90, 889-905.
Elkins, C. A., & Nikaido, H. (2002) J Bacteriol 184, 6490-6498.
Gibbons, S., Oluwatuyi, M, Kaatz, G. W. (2003) J Antimicrob Chemother 51, 13-17.
Gomer, C. J. (1991) Photochem Photobiol 54, 1093-1107.
Gonzalez-Pasayo, R., and E. Martinez-Romero (2000) Mol Plant Microbe Interact 13, 572-577.
Gross, S., Brandis, A., Chen, L., Rosenbach-Belkin, V., Roehrs, S., Scherz, A. & Salomon, Y. (1997) Photochem Photobiol 66, 872-878.
Hamblin, M. R., & E. L. Newman (1994) J Photochem Photobiol B 23, 3-8.
Hamblin, M. R., Zahra, T., Contag, C. H., McManus, A. T. & Hasan, T. (2003) The Journal of infectious diseases. 187, 1717-1726.
Harrison, A. P., Hafner, J. L. & O'Bryan, C. (1972) Mutat Res 14, 447-449.
Hass, B. S., & Webb, R. B (1981) Mutat Res 81, 277-285.
Hass, B. S., & Webb, R. B. (1979) Mutat Res 60, 1-11.
Hausmann, W. (1908) Wien. Klin. Wochnschr 21, 1527-1529.
Henney, J. E. (2000) Jama 283, 2779.
Hongcharu, W., Taylor, C. R., Chang, Y., Aghassi, D., Suthamjariya, K. & Anderson, R. R. (2000) J Invest Dermatol 115, 183-192.
Houba-Herin, N., Calberg-Bacq, C. M., Piette, J. & Van de Vorst, A. (1982) Photochem Photobiol 36, 297-306.
Hsieh, P. C., Siegel, S. A., Rogers, B., Davis, D., Lewis, K. (1998) Proc Natl Acad Sci USA 95, 6602-6606.
Imray, F. P., & MacPhee, D. G. (1973) Mol Gen Genet. 123, 289-298.
Jacob, H. E. (1971) Photochem Photobiol 14, 743-745.
Jacob, H. E., & Hamann, M. (1975) Photochem Photobiol 22, 237-241.
Jacob, H. E., Fleck, W. & Strauss, D. (1977) Z Allg Mikrobiol 17, 569-573.
Jenkins, M. P., Buonaccorsi, G. A., Raphael, M., Nyamekye, I., McEwan, J. R., Bown, S. G. & Bishop, C. C. (1999) Br J Surg 86, 1258-1263.
Jennings, B. R., & Ridler P. J. (1983) Biophys Struct Mech 10, 71-79.
Jesionek, A., & von Tappenier, H. (1903) Muench. Med. Wochneshr 47, 2042.
Jett, B. D., Hatter, K. L., Huycke, M. M. & Gilmore, M. S. (1997) Biotechniques 23, 648-650.
Kaatz, G. W., Moudgal, V. V., Seo, S. M., Kristiansen, J. E. (2003) Antimicrob Agents Chemother 47, 719-726.
Kaatz, G. W., Seo, S. M., O'Brien, L., Wahiduzzaman, M., Foster, T. J. (2000) Antimicrob Agents Chemother 44, 1404-1406.
Kolaczkowski, M., Michalak, K, Motohashi, N. (2003) Int J Antimicrob Agents 22, 279-283.
Krupka, R. (1999) J Membr Biol 172, 129-143.
Kubin, A., Wierrani, F., Jindra, R. H., Loew, H. G., Grunberger, W., Ebermann, R. & Alth, G. (1999) Drugs Exp Clin Res 25, 13-21.
Lage, H. (2003) Int J Antimicrob Agents 22, 188-199.
Lee, B. D., Li, Z., French, K. J., Zhuang, Y., Xia, Z., Smith, C. D. (2004) J Med Chem 47, 1413-1422.
Lewis, K. (1999) Current Biology 9, 403-407.
Lewis, K. (2001) J Mol Microbiol Biotechnol 2, 247-254.
Li, X. Z., L. Zhang, R. Srikumar, and K. Poole. (1998) Antimicrob. Agents Chemother 42, 399-403.
Liu, Z. L., Hirano, T., Tanaka, S., Onda, K., Oka, K. (2003) J Pharm Pharmacol 55, 1531-1537.
Lomovskaya, O., & Lewis, K. (1992) Proceedings of the National Academy of Sciences of the United States of America 89, 8938-8942.
Lomovskaya, O., Warren, M. S., Lee, A., Galazzo, J., Fronko, R., Lee, M., Blais, J., Cho, D., Chamberland, S., Renau, T., Leger, R., Hecker, S., Watkins, W., Hoshino, K., Ishida, H., Lee, V. J. (2001) Antimicrob Agents Chemother 45, 105-116.
Lu, X. M., Fischman, A. J., Stevens, E., Lee, T. T., Strong, L., Tompkins, R. G. & Yarmush, M. L. (1992) J Immunol Methods 156, 85-99.
Malik, Z., Ladan, H. & Nitzan, Y. (1992) J. Photochem. Photobiol. B 14, 262-266.
Malik, Z., Ladan, H., Nitzan, Y. & Ehrenberg, B. (1990) J. Photochem. Photobiol. B. 6, 419-430.
Mao, W., Warren, M. S., Black, D. S., Satou, T., Murata, T., Nishino, T., Gotoh, N., Lomovskaya, O. (2002) Mol Microbiol 46, 889-901.
Markham, P. N., Westhaus, E., Klyachko, K., Johnson, M. E., Neyfakh, A. A. (1999) Antimicrob Agents Chemother 43, 2404-2408.
Martin, J. P., & Logsdon, N. (1987) J Biol Chem. 262, 7213-7219.
Masip, I., Ferrandiz-Huertas, C., Garcia-Martinez, C., Ferragut, J. A., Ferrer-Montiel, A., Messeguer, A. (2004) J Comb Chem 6, 135-141.
Merchat, M., Bertolini, G., Giacomini, P., Villanueva, A. & Jori, G (1996a) J. Photochem. Photobiol. B 32, 153-157.
Merchat, M., Spikes, J. D., Bertoloni, G. & Joni, G. (1996b) J. Photochem. Photobiol. B 35, 149-157.
Millson, C. E., Wilson, M., Macrobert, A. J., Bedwell, J. & Bown, S. G. (1996) J. Med. Microbiol. 44, 245-252.
Minnock, A., Vernon, D. I., Schofield, J., Griffiths, J., Parish, J. H. & Brown, S. B. (1996) J. Photochem. Photobiol. B 32, 159-164.
Minnock, A., Vernon, D. I., Schofield, J., Griffiths, J., Parish, J. H. & Brown, S. B. (2000) Antimicrob Agents Chemother 44, 522-527.
Mitchell, B. A., Brown, M. H., Skurray, R. A. (1998) Antimicrob Agents Chemother 42, 475-477.
Morrissey, J. P., & Osbourn, A. E. (1999) Microbiol Mol Biol Rev 63, 708-724.
Murata, T., Kuwagaki, M., Shin, T., Gotoh, N., Nishino, T. (2002) Biochem Biophys Res Commun 299, 247-251.
Musumeci, R., Speciale, A., Costanzo, R., Annino, A., Ragusa, S., Rapisarda, A., Pappalardo, M. S., Lauk, L. (2003) Int J Antimicrob Agents 22, 48-53.
Nikaido, H. (1999) J Bacteriol 181, 4-8.
Nitzan, Y., Balzam-Sudakevitz, A. & Ashkenazi, H. (1998) J Photochem Photobiol B, 42, 211-218.
Nitzan, Y., Gutterman, M., Malik, Z. & Ehrenberg, B. (1992) Photochem. Photobiol. 55, 89-96.
Norton, S. (1994) J Am Acad Dermatol 31, 683-686.
Ochsner, M. (1997) J Photochem Photobiol B 39, 1-18.
Palumbo, J. D., C. I. Kado, and D. A. Phillips (1998) Journal of Bacteriology 180, 3107-3113.
Paulsen, I. T., Chen, J., Nelson, K. E., and M. H. Saier Jr (2002) Comparative genomics of microbial drug efflux systems. In Microbial Multidrug Efflux. Lewis, I. T. P. a. K. (ed.) Norfolk: Horizon Press, pp. 5-21.
Perlin, M., Mao, J. C., Otis, E. R., Shipkowitz, N. L. & Duff, R. G. (1987) Antiviral Res 7, 43-51.
Pleban, K., Hoffer, C., Kopp, S., Peer, M., Chiba, P., Ecker, G. F. (2004) Arch Pharm (Weinheim) 337, 328-334.
Raab, C. (1900) Z. Biol. 39, 524-546.
Ranganathan, K. R., Seshadri, T. R. (1974) Indian J Chem 12, 993.
Redmond, R. W., & Gamlin, J. N. (1999) Photochem Photobiol 70, 391-475.
Renau, T. E., Leger, R., Flamme, E. M., Sangalang, J., She, M. W., Yen, R., Gannon, C. L., Griffith, D., Chamberland, S., Lomovskaya, O., Hecker, S. J., Lee, V. J., Ohta, T., Nakayama, K. (1999) J Med Chem 42, 4928-4931.
Rivoltini, L., Colombo, M. P., Supino, R., Ballinari, D., Tsuruo, T., Parmiani, G. (1990) Int J Cancer 46, 727-732.
Rockson, S. G., Lorenz, D. P., Cheong, W. F. & Woodburn, K. W. (2000) Circulation 102, 591-596.
Rogers, B., A. Decottignies, M. Kolaczkowski, E. Carvajal, E. Balzi, and A. Goffeau (2001) J Mol Microbiol Biotechnol 3, 207-214.
Schafer, M., Schmitz, C., Facius, R., Horneck, G., Milow, B., Funken, K. H. & Ortner, J. (2000) Photochem Photobiol, 71, 514-523.
Schmeller, T., Latz-Bruning, B., Wink, M. (1997) Phytochemistry 44, 257-266.
Slapak, C. A., Dahlheimer, J., Piwnica-Worms, D. (2001) J Clin Pharmacol.
Sorokin, A. (2004) Curr Pharm Des 10, 647-657.
Stermitz, F. R., Cashman, K. K., Halligan, K. M., Morel, C., Tegos, G. P., Lewis, K. (2003) Bioorg Med Chem Lett 13, 1915-1918.
Stermitz, F. R., Lorenz, P., Tawara, J. N., Zenewicz, L. A., & Lewis, K. (2000a) Proc Natl Acad Sci USA 97, 1433-1437.
Stermitz, F. R., Scriven, L. N., Tegos, G., Lewis, K. (2002) Planta Med 68, 1140-1141.
Stermitz, F. R., Tawara-Matsuda, J, Lorenz, P., Mueller, P., Zenewicz, L., Lewis, K. (2000b) J Nat Prod 63, 1146-1149.
Stoodley, L. H., J. W. Costerton, P., Stoodley (2004) Nature Reviews Microbiology 2, 95-108.
Strong, L., Yarmush, D. M. & Yarmush, M. L. (1994) Ann N Y Acad. Sci. 745, 297-320.
Tegos, G., Stermitz, F. R., Lomovskaya, O., & Lewis, K. (2002) Antimicrob Agents Chemother 46, 3133-3141.
Trauner, K. B., & Hasan, T. (1996) Photochem Photobiol 64, 740-750.
Valduga, G., Breda, B., Giacometti, G. M., Joni, G. & Reddi, E. (1999) Biochem Biophys Res Commun 256, 84-88.
Van Bambeke, F., Balzi, E., & P. M. Tulkens (2000) Biochem Pharmacol 60, 457-470.
Vazquez-Laslop, N., Zheleznova, E. E., Markham, P. N., Brennan, R. G., Neyfalch, A. A. (2000) Biochem Soc Trans 28, 517-520.
Velezheva, V. S., Brennan, P. J., Marshakov, V. Y., Gusev, D. V., Lisichkina, I. N., Peregudov, A. S., Tchernousova, L. N., Smirnova, T. G., Andreevskaya, S. N., Medvedev, A. E. (2004) J Med Chem 47, 3455-3461.
Von Tappeiner, H., & Jodlbauer, A. (1904) Disch. Arch. Klin. Med. 80, 422-487.
Wainwright, M. (2003) Biotech Histochem 78, 147-155.
Wang, E. J., Barecki-Roach, M, & Johnson, W. W. (2004) J Pharm Pharmacol 56, 123-128.
Wang, E. J., Casciano, C. N., Clement, R. P., Johnson, W. W. (2001) Biochem Biophys Res Commun 289, 580-585.
Wilson, M. (1993) J. Appl. Bacteriol. 75, 299-306.
Wilson, M. P., J. (1995) Lasers Surg Med 16, 272-276.
Ziebell, R., Imray, F. P. & Macphee, D. G. (1977) J Gen Microbiol 101, 143-149.

What is claimed is:

1. A method of inactivating microorganisms comprising contacting the microorganism with a phenothiazinium and a microbial MDR inhibitor and irradiating the phenothiazinium such that a phototoxic species is produced that inactivates the microorganism.

2. The method of claim 1, wherein the microorganism is selected from the group consisting of bacteria, fungus, protozoa, virus, parasite and yeast.

3. The method of claim 2, wherein the bacteria is of a genus selected from the group consisting of Staphylococcus, Streptococcus, Enterococcus, Mycobacterium, Pseudomonas, Salmonella, Shigella, Escherichia, Erwinia, Klebsiella, Borrelia, Treponema, Campylobacter, Helicobacter, Bordetella, Neisseria, Legionella, Leptospira, Serpulina, Mycoplasma, Bacteroides, Klebsiella, Yersinia, Chlamydia, Vibrio, Actinobacillus, Porphyria, Hemophilus, Helicobacter, Pasteurella, Pseudomonas, Peptostreptococcus, Listeria, Propionibacterium, Mycobacterium, Corynebacterium and Dermatophilus.

4. The method of claim 1, wherein the microorganism is a virus selected from the group consisting of HIV, Hepatitis virus, Influenza virus, Rhinovirus, Papilloma virus, Measles virus, Herpes virus, Rotavirus, Parvovirus, Psittacosis virus, and Ebola virus.

5. The method of claim 1, wherein the microorganism to be inactivated is in or on a living animal.

6. The method of claim 5 wherein the microorganism to be inactivated is located on the skin or mucous membranes of the living animal, or within wounds, cuts or abrasions of the living animal.

7. The method of claim 5, wherein the living animal is a human.

8. The method of claim 1, wherein the microorganism to be inactivated is located in or on an inanimate object or substance.

9. The method of claim 1, wherein the microbial MDR inhibitor is selected from the group consisting of INF271, MC₂₀₇₁₁₀, 5′ Methoxyhydnocarpin, Pheophorbide a, Chrysoplenol D, Chrysoplenetin, Genistein, Biochanin, Polyacylated Neohesperidosides, 4′,6′-Dihydroxy-3′,5′ dimethyl-2′-methoxychalcone, 3,5-Dimethoxy-4′-hydroxy-trans-stilbene, 3,5,4′-Trimethoxy-trans-stilbene, Difluorocyclopropyl quinoline, Dihydropyrroloquinolines, GG918, Verpamil Pgp, Cyclosporins Pgp, Reserpine Pgp, Propafenone Pgp, Pyridazino[4,3-b]indoles Pgp, Hypericin, Cyclooxygenase-2,3-Oxopiperazinium and Perhydro-3-oxo-1,4-diazepinium derivatives, Tetrandrine, Phenothiazines and mixtures thereof.

10. The method of claim 1, wherein the phenothiazinium is selected from the group consisting of toluidine blue derivatives, toluidine blue O, methylene blue, new methylene blue N, new methylene blue BB, new methylene blue FR, 1,9-dimethylmethylene blue chloride, methylene blue derivatives, methylene green, methylene violet Bernthsen, methylene violet 3RAX, Nile blue, Nile blue derivatives, malachite green, Azure blue A, Azure blue B, Azure blue C, safranine O, neutral red, 5-ethylamino-9-diethylaminobenzo[a]phenothiazinium chloride, 5-ethylamino-9-diethylaminobenzo[a]phenoselenazinium chloride, thiopyronine, thionine, and mixtures thereof.

11. The method of claim 1, wherein the phenothiazinium is methylene blue.

12. The method of claim 1, wherein the phenothiazinium is toluidine blue O.

13. The method of claim 10, wherein the microorganism is contacted with a composition comprising the phenothiazinium.

14. The method of claim 13, wherein the composition further comprises a member selected from the group consisting of a microbial MDR inhibitor, a pharmaceutically acceptable carrier, an excipient, an antibiotic, an antimicrobial agent, a disinfectant, and a detergent.

15. The method of claim 1, wherein the method further comprises contacting the microorganism with an antibiotic, an antimicrobial agent, a disinfectant, or a detergent.

16. The method of claim 1, wherein the microorganism is contacted with the phenothiazinium and the microbial MDR inhibitor at the same time.

17. The method of claim 1, wherein the microorganism is contacted with the phenothiazinium before it is contacted with the microbial MDR inhibitor.

18. The method of claim 15, wherein the microorganism is contacted with the phenothiazinium after it is contacted by the microbial MDR inhibitor.

19. The method of claim 13, wherein the composition comprises a liquid, cream, or lotion.

20. The method of claim 13, wherein the composition comprises a liquid spray.

21. The method of claim 13 wherein the composition comprises an aerosol spray.

22. The method of claim 1, wherein the irradiation is provided by a light source that emits light at a wavelength in the range of about 450 to about 750 nm

23. The method of claim 1, wherein the irradiation is provided by a light source that emits light at fluence in the range of about 10 to about 1000 J/cm²

24. The method of claim 1, wherein the irradiation is provided by a light source that emits light at wavelength in the range of about 450 to about 750 nm and a fluence in the range of about 10 to about 1000 J/cm².

25. The method of claim 1, wherein the irradiation is provided by a lamp, a laser or a fiber optic device.

26. A method of treating a subject infected with a microorganism, said method comprising the steps of administering a phenothiazinium and a microbial MDR inhibitor to the subject, irradiating the phenothiazinium such that a phototoxic species is produced that inactivates the microorganism, thereby treating the subject.

27. The method of claim 26, wherein the microorganism is selected from the group consisting of bacteria, fungus, protozoa, virus, parasite and yeast.

28. The method of claim 27, wherein the bacteria is of a genus selected from the group consisting of Staphylococcus, Streptococcus, Enterococcus, Mycobacterium, Pseudomonas, Salmonella, Shigella, Escherichia, Erwinia, Klebsiella, Borrelia, Treponema, Campylobacter, Helicobacter, Bordetella, Neisseria, Legionella, Leptospira, Serpulina, Mycoplasma, Bacteroides, Klebsiella, Yersinia, Chlamydia, Vibrio, Actinobacillus, Porphyria, Hemophilus, Helicobacter, Pasteurella, Pseudomonas, Peptostreptococcus, Listeria, Propionibacterium, Mycobacterium, Corynebacterium and Dermatophilus.

29. The method of claim 26, wherein the microorganism is a virus selected from the group consisting of HIV, Hepatitis virus, Influenza virus, Rhinovirus, Papilloma virus, Measles virus, Herpes virus, Rotavirus, Parvovirus, Psittacosis virus, and Ebola virus.

30. The method of claim 26, wherein the microorganism to be inactivated is located on the skin or mucous membranes of the subject, or within wounds, cuts or abrasions of the subject.

31. The method of claim 26, wherein the subject is a human.

32. The method of claim 26, wherein the microbial MDR inhibitor is selected from the group consisting of INF271, MC₂₀₇₁₁₀, 5′ Methoxyhydnocarpin, Pheophorbide a, Chrysoplenol D, Chrysoplenetin, Genistein, Biochanin, Polyacylated Neohesperidosides, 4′,6′-Dihydroxy-3′,5′ dimethyl-2′-methoxychalcone, 3,5-Dimethoxy-4′-hydroxy-trans-stilbene, 3,5,4′-Trimethoxy-trans-stilbene, Difluorocyclopropyl quinoline, Dihydropyrroloquinolines, GG918, Verpamil Pgp, Cyclosporins Pgp, Reserpine Pgp, Propafenone Pgp, Pyridazino[4,3-b]indoles Pgp, Hypericin, Cyclooxygenase-2,3-Oxopiperazinium and Perhydro-3-oxo-1,4-diazepinium derivatives, Tetrandrine, Phenothiazines and mixtures thereof.

33. The method of claim 26, wherein the phenothiazinium is selected from the group consisting of toluidine blue derivatives, toluidine blue O, methylene blue, new methylene blue N, new methylene blue BB, new methylene blue FR, 1,9-dimethylmethylene blue chloride, methylene blue derivatives, methylene green, methylene violet Bernthsen, methylene violet 3RAX, Nile blue, Nile blue derivatives, malachite green, Azure blue A, Azure blue B, Azure blue C, safranine O, neutral red, 5-ethylamino-9-diethylaminobenzo[a]phenothiazinium chloride, 5-ethylamino-9-diethylaminobenzo[a]phenoselenazinium chloride, thiopyronine, thionine, and mixtures thereof.

34. The method of claim 1, wherein the phenothiazinium is methylene blue.

35. The method of claim 1, wherein the phenothiazinium is toluidine blue O.

36. The method of claim 33, wherein a composition comprising the photosensitizer is administered to the subject.

37. The method of claim 36, wherein the composition further comprises a member selected from the group consisting of a microbial MDR inhibitor, a pharmaceutically acceptable carrier, an excipient, an antibiotic, an antimicrobial agent, a disinfectant, and a detergent.

38. The method of claim 26, wherein the method further comprises administering an antibiotic or an antimicrobial agent.

39. The method of claim 26, wherein the microbial MDR inhibitor is administered at the same time as the phenothiazinium.

40. The method of claim 26, wherein the microbial MDR inhibitor is administered before the phenothiazinium.

41. The method of claim 26, wherein the microbial MDR inhibitor is administered after the phenothiazinium.

42. The method of claim 36, wherein the composition comprises a liquid, cream, or lotion.

43. The method of claim 36, wherein the composition comprises a liquid spray.

44. The method of claim 36, wherein the composition comprises an aerosol spray.

45. The method of claim 26, wherein the irradiation is provided by a light source that emits light at wavelength in the range of about 450 to about 750 nm.

46. The method of claim 26, wherein the irradiation is provided by a light source that emits light at fluence in the range of about 10 to about 1000 J/cm².

47. The method of claim 26, wherein the irradiation is provided by a light source that emits light at wavelength in the range of about 450 to about 750 nm and a fluence in the range of about 10 to about 1000 J/cm².

48. The method of claim 26, wherein the irradiation is provided by a lamp, a laser or a fiber optic device.

49. The method of claim 1, further comprising obtaining the phenothiazinium.

50. The method of claim 1, further comprising synthesizing the phenothiazinium.

51. The method of claim 13, further comprising obtaining the composition.

52. The method of claim 13, further comprising synthesizing the composition.

53. The method of claim 26, wherein the step of administering comprises topical application of the phenothiazinium or the microbial MDR inhibitor.

54. The method of claim 26, wherein the step of administering comprises inhalation of the phenothiazinium or the microbial MDR inhibitor.

55. The method of claim 26, wherein the step of administering comprises ingestion of the phenothiazinium or the microbial MDR inhibitor.

56. The method of claim 26, wherein the step of administering comprises injection of the phenothiazinium or the microbial MDR inhibitor.

57. The method of claim 26, wherein the step of administering comprises implantation of the phenothiazinium or the microbial MDR inhibitor.

58. A kit for inactivating microorganisms comprising a phenothiazinium, a microbial MDR inhibitor and directions for use.

59. The kit of claim 58, further comprising means for irradiating the microorganism.

60. A kit for treating a subject contaminated with bacterial spores comprising a photosensitizer and instructions for use.

61. The kit of claim 60, further comprising means for irradiating the subject.

62. The kit of claim 60, wherein the phenothiazinium is present in a composition comprising a therapeutically effective amount of the phenothiazinium.
